(19) United States
(12) Patent Application Publication
(10) Pub. No.: US 2004/0266766 A1
(43) Pub. Date: Dec. 30, 2004

(54) Selective urokinase inhibitors

(75) Inventor: Stefan Sperl, Munchen (DE)

Correspondence Address: ROTHWELL, FIGG, ERNST & MANBECK, P.C., 1425 K STREET, N.W., SUITE 800, WASHINGTON, DC, 20005, US

(73) Assignee: Wilex AG, Munchen (DE)

(21) Appl. No.: 10/866,196

(22) Filed: Jun. 14, 2004

Related U.S. Application Data
(63) Continuation-in-part of application No. PCT/EP02/14157, filed on Dec. 12, 2002.

(30) Foreign Application Priority Data
Dec. 12, 2001 (DE) 101 61 062.9
Sep. 2, 2002 (DE) 102 40 452.6

(51) Int. Cl. A61K 31/538; A61K 31/517; A61K 31/47

(52) U.S. Cl. 514/230.5; 514/266.4; 514/309; 514/312

(57) ABSTRACT

The present invention concerns new selective inhibitors of the urokinase plasminogen activator (uPA, EC 3.4.21.31) and their use as therapeutic agents for treating urokinase-associated diseases such as malignant tumors and the formation of metastases.

The present invention concerns new selective inhibitors of the urokinase plasminogen activator (uPA, EC 3.4.21.31) and their use as therapeutic agents for treating urokinase-associated diseases such as malignant tumors and formation of metastases. The invention especially concerns new highly selective and highly active inhibitors of the urokinase plasminogen activator (uPA, EC 3.4.21.31) of the aryl-guanidine type.

The plasminogen activator of the urokinase type (uPA) plays a key role in tumor invasion and the formation of metastases (Schmitt et al., J. Obst. Gyn. 21 (1995), 151-165. uPA is overexpressed in various types of tumor cells (Kwaan, Cancer Metastasis Rev. 11 (1992), 291-311) and binds to the tumor-associated uPA receptor (uPA-R) where plasminogen is activated to plasmin. Plasmin is able to degrade various components of the extracellular matrix (ECM) such as fibronectin, laminin and collagen type IV. It also activates some other ECM-degrading enzymes, in particular matrix metalloproteinases. High amounts of tumor-associated uPA correlate with a higher risk of forming metastases for cancer patients (Stephens et al., Breast Cancer Res. & Treat. 52 (1998), 99-111). Hence an inhibition of the proteolytic activity of uPA is a good approach for an anti-metastatic therapy.

A common feature of many of the known synthetic uPA inhibitors is a basic residue which contains amidino or guanidino groups and can bind to Asp¹⁸⁹ in the S1 specificity pocket of uPA and acts there as an arginine mimetic (Spraggon et al., Structure 3 (1995), 681-691).

However, most of the known inhibitors are not selective for uPA but also inhibit other serine proteases such as trypsin, thrombin, plasmin or tissue plasminogen activator (tPA).

p-Aminobenzamidine is a moderately selective uPA inhibitor with an inhibition constant of 82 μM. Billstroem et al. (Int. J. Cancer 61 (1995), 542-547) showed that there was a considerable decrease in the growth rate of DU145 tumors (a prostate adenocarcinoma cell line) in SCID mice when p-aminobenzamidine was administered orally at a daily dose of 125 to 250 mg/kg/day. The side-effects were negligibly small.

Some monosubstituted phenylguanidines have proven to be effective and selective uPA inhibitors in vitro. These small molecules have inhibition constants in the micromolar range but they only bind in the S1 pocket of uPA (Yang et al., J. Med. Chem. 33 (1990), 2956-2961). No biological investigations have been carried out with these compounds.

The diuretic amiloride is a selective uPA inhibitor (Kᵢ, uPA=7 μM) which prevents the formation of lung metastases after i.v. inoculation of rat breast adenocarcinoma cells (Kellen et al., Anticancer Res. 8 (1988), 1373-1376). Some derivatives of 3-amidino-phenylalanine have also proven to be effective inhibitors of serine proteases, but these compounds generally only have a low selectivity for uPA (Stürzebecher et al., J. Med. Chem. 40 (1997), 3091-3099; Stürzebecher et al., J. Enzyme Inhib. 9 (1995), 87-99).

Known uPA inhibitors are derivatives of benzo[b]-thiophene-2-carboxamidine (B428 and B623: Kᵢ, uPA=0.32 and 0.07 μM respectively; U.S. Pat. No. 5,340,833). Rabbani et al., (Int. J. Cancer 63 (1995), 840-845) and Xing et al. (Cancer Res. 57 (1997), 3585-3593) showed that there was a decrease in tumor growth and formation of metastases in a syngenic model for rat prostate carcinoma and mouse mammary carcinoma after administration of 4-iodo-benzo[b]-thiophene-2-carboxamidine (B428). The latter investigations showed a further decrease of primary tumor growth when B428 was administered together with the anti-oestrogen Tamoxifen.

The German Patent Application 199 40 389.9 proposes the use of arylguanidine and in particular phenylguanidine derivatives as selective uPA inhibitors. These compounds contain a further substituent on the aromatic ring system preferably in the para position relative to the guanidine group which contains an optionally substituted methylene group followed by hydrogen donor/acceptor functionalities.

Due to this substitution pattern the compounds have a particularly high effectivity and selectivity for uPA. These compounds are assumed to interact as an arginine mimetic with the amino acid residue Asp¹⁸⁹ in the S1 pocket of uPA and to interact with the S2 and/or S3 pocket of uPA.

The German Patent Application 100 13 715.6 describes further arylguanidine derivatives which can interact even more specifically with uPA and especially with the amino acid residues Gln¹⁹² and/or Ser²¹⁴. In addition to the guanidine group, these compounds contain a further substituent on the aromatic ring system which contains an optionally substituted methylene group followed by a hydrogen donor, a hydrogen acceptor and again a hydrogen donor functionality.

The international Patent Application WO 00/04954 describes arylamidine derivatives and in particular amidinophenylalanine derivatives as urokinase inhibitors.

An object of the present invention was to provide new highly selective and highly active inhibitors of the urokinase plasminogen activator.

This object is achieved according to the invention by the use of compounds of the general formula I

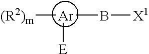

in which

Ar denotes an aromatic or heteroaromatic ring system,

E denotes

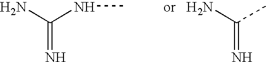

or Ar and E together form a residue

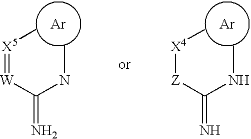

in which Z can be O, NH or C═O and X⁴ can be C═O, HN or CH₂ and W can be N, CR³ or CR⁶ and X⁵ can be CH, CR³, CR⁶ or N,

B denotes —SO₂—, —CR³₂—, —NR³— or —NH—,

X¹ denotes NR¹³R⁴, OR³, SR³, COOR³, CONR³R⁴ or COR⁵,

R¹ denotes H, an optionally substituted alkyl, alkenyl, alkinyl, aryl, heteroaryl residue or COOR³, CONR³R⁴ or COR⁵,

R² denotes halogen, C(R⁶)₃, C₂(R⁶)₅, OC(R⁶)₃ or OC₂(R⁶)₅,

R³ in each case independently denotes H or any organic residue,

R¹³ denotes a group of the general formula (IIa) or (IIb),

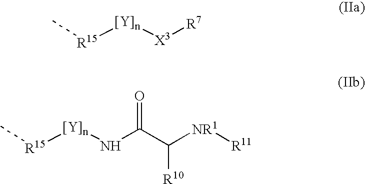

X² denotes NH, NR⁴, O or S,

X³ denotes NH, NR⁴, O, S, CO, COO, CONH or CONR⁴,

Y denotes C(R⁸)₂, NH or NR³,

R⁴ denotes H or a branched or unbranched, optionally substituted alkyl, alkenyl or alkinyl residue,

R⁵ denotes H, an alkyl, alkenyl, alkinyl, carboxy-alkyl, carboxy-alkenyl, carboxyl-alkinyl, carboxy-aryl, carboxy-heteroaryl, —(CO)NR³R⁴ or —COO—R³ in which the alkyl, aryl and heteroaryl residues can optionally be substituted,

R⁶ is in each case independently H or halogen and in particular F,

R⁷ denotes H or an optionally substituted alkyl, alkenyl, alkinyl, aryl or heteroaryl residue or —COR⁹,

R⁸ in each case independently denotes H, or a branched or unbranched, optionally substituted alkyl, alkenyl, alkinyl, aryl, heteroaryl, aralkyl, alkylaryl, heteroaralkyl residue or/and a substituted or unsubstituted bicyclic or polycyclic residue,

R⁹ denotes H or a branched or unbranched, optionally substituted alkyl, alkenyl, alkinyl, aryl or/and heteroaryl residue,

R¹⁰ denotes a residue (C(R¹)₂)ₙ—X³R⁵, in particular —CH₂—OH,

R¹¹ denotes H, a carbonyl residue —CO—R¹², a carbonamido residue —CONR¹²₂, an oxycarbonyl residue —COO—R¹² or particularly preferably a sulfonyl residue —SO₂R¹²,

R¹² denotes H, a branched or unbranched, substituted or unsubstituted alkyl, alkenyl, alkinyl, aryl or heteroaryl residue or a substituted or unsubstituted cyclic alkyl residue or a substituted or unsubstituted aralkyl, alkylaryl or heteroaralkyl residue or a substituted or unsubstituted bicylic or polycyclic residue,

R¹⁵ represents C═X², NR³ or CR³₂,

n is an integer from 0 to 2,

m is an integer from 0 to 5,

o is an integer from 1 to 5,

p is an integer from 1 to 5.

or salts of these compounds to produce an agent for inhibiting the urokinase plasminogen activator.

Compounds of the general formula III are preferred

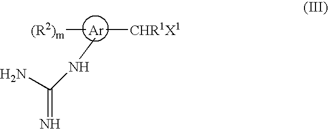

in which

Ar denotes an aromatic or heteroaromatic ring system,

X¹ denotes NR¹³R⁴, OR³, SR³, COOR³, CONR³R⁴ or COR⁵,

R¹ denotes H, an optionally substituted alkyl, alkenyl, alkinyl, aryl, heteroaryl residue or COOR³, CONR³R⁴ or COR⁵,

R² denotes halogen, C(R⁶)₃, C₂(R⁶)₅, OC(R⁶)₃ or OC₂(R⁶)₅,

R³ denotes H or any organic residue,

R¹³ denotes a group of the general formula (IVa) or (IVb),

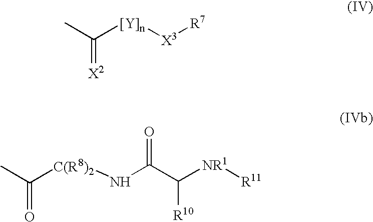

X² denotes NH, NR⁴, O or S,

X³ denotes NH, NR⁴, O, S, CO, COO, CONH or CONR⁴,

Y denotes C(R⁸)₂, NH or NR³.

R⁴ denotes H or a branched or unbranched, optionally substituted alkyl, alkenyl or alkinyl residue,

R⁵ denotes H, an alkyl, alkenyl, alkinyl, carboxy-alkyl, carboxy-alkenyl, carboxyl-alkinyl, carboxy-aryl, carboxy-heteroaryl, —(CO)NR³R⁴ or —COO—R³ in which the alkyl, aryl and heteroaryl residues can optionally be substituted,

R⁶ is in each case independently H or halogen and in particular F,

R⁷ denotes H or an optionally substituted alkyl, alkenyl, alkinyl, aryl or heteroaryl residue or —COR⁹,

R⁸ in each case independently denotes H, halogen, or a branched or unbranched, optionally substituted alkyl, alkinyl, aryl, heteroaryl residue or/and (CH₂)ₘ—OH,

R⁹ denotes H or a branched or unbranched, optionally substituted alkyl, alkenyl, alkinyl, aryl or/and heteroaryl residue,

R¹⁰ denotes a residue (C(R¹)₂)ₒ—X³R⁵, in particular —CH₂—OH,

R¹¹ denotes H, a carbonyl residue —CO—R¹², an oxycarbonyl residue —COO—R¹² or particularly preferably a sulfonyl residue —SO₂R¹²,

R¹² denotes a branched or unbranched, substituted or unsubstituted alkyl, alkenyl, alkinyl, aryl or heteroaryl residue or a substituted or unsubstituted cyclic alkyl residue or a substituted or unsubstituted aralkyl, alkylaryl or heteroaralkyl residue or a substituted or unsubstituted bicylic or polycyclic residue,

n is an integer from 0 to 2,

m is an integer from 0 to 5,

o is an integer from 1 to 5,

or salts of these compounds to produce an agent for inhibiting the urokinase plasminogen activator.

The compounds can be present as salts, preferably as physiologically acceptable acidic salts e.g. as salts of mineral acids, particularly preferably as hydrochlorides or as salts of suitable organic acids. The guanidinium group can optionally carry protective functions that can be preferably cleaved under physiological conditions. The compounds can be present as optically pure compounds or as mixtures of enantiomers or/and diastereoisomers.

The ring system Ar preferably contains 4 to 30 and in particular 5 to 10 C-atoms. In the compounds of the general formula (I) or (III), Ar is preferably an aromatic or heteroaromatic ring system with one ring. Compounds are also preferred in which Ar and E together form a bicyclic system. Heteroaromatic systems preferably contain one or more O, S or/and N atoms. A preferred aromatic ring system is a benzene ring; preferred heteroaromatic ring systems are pyridinyl, pyrimidinyl or pyrazinyl, especially with nitrogen at position 2. Preferred bicyclic ring systems are those with nitrogen or oxygen at positions Z or W. Ar is most preferably a benzene ring.

Compounds with the following structural elements are particularly preferred

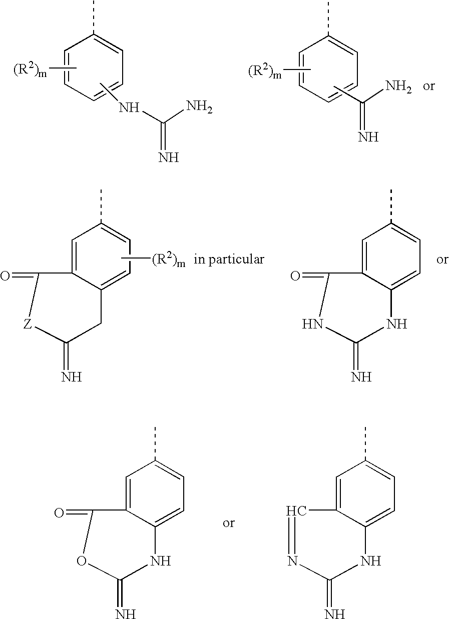

In the compounds of the general formula (I) or (III), the substituents B e.g. CHX¹R¹ and E, e.g. NHC(NH)NH₂ (guanidino) or NH₂CNH (amidino) in the ring system Ar are preferably in the meta or para position and particularly preferably in the para position relative to one another. Moreover, Ar can contain one or more additional substituents R² that are different from hydrogen. The number of substituents R² is preferably 0, 1, 2 or 3, particularly preferably 0 or 1 and most preferably 0. R² can denote halogen, C(R⁶)₃, C₂(R⁶)₅, OC(R⁶)₃ or OC₂(R⁶)₅ in which case R⁶ is in each case independently H or halogen and in particular F. Preferred examples of R 2 are halogen atoms (F, Cl, Br or I), CH₃, CF₃, OH, OCH₃ or OCF₃.

The compounds according to the invention contain a guanidino group and are characterized by a high selectivity. For this reason E is often preferably —NH—C(NH)—NH₂.

The substituent B in formula (I) or —CHX¹R¹ in formula (III) is important for the inhibitor activity. B is preferably selected from —SO₂—, —NR³—, —NH— or/and —CR³₂—, in particular —CR¹₂—.

R¹ can be H or an optionally substituted alkyl, alkenyl, alkinyl, aryl or/and heteroaryl residue or COOR³, CONR³R⁴ or COR⁵. R¹ is most preferably H.

If not stated otherwise an alkyl residue as used herein is preferably a straight-chained or branched C₁-C₃₀ alkyl group, preferably a C₁-C₁₀ alkyl group, in particular a C₁-C₄ alkyl group or a C₃-C₃₀ cycloalkyl group in particular a C₃-C₈ cycloalkyl group that can for example be substituted with C₁-C₃ alkoxy, hydroxyl, carboxyl, amino, sulfonyl, nitro, cyano, oxo or/and halogen and also with aryl or heteroaryl residues. If not stated otherwise, alkenyl and alkinyl residues are herein preferably C₂-C₁₀ groups, in particular C₂-C₄ groups which can optionally be substituted as previously stated. Aryl and heteroaryl residues can for example be substituted with C₁-C₆ alkyl, C₁-C₃ alkoxy-hydroxyl, carboxyl, sulfonyl, nitro, cyano or/and oxo. Aryl and heteroaryl residues preferably contain 3 to 30, in particular 4 to 20, preferably 5 to 15 and most preferably 6 to 10 C atoms.

X¹ preferably represents NR¹³R⁴.

R³ can denote H or any organic residue. The organic residue is in particular a residue with 1 to 30 carbon atoms. This residue can be saturated or unsaturated, linear, branched or cyclic and optionally contain substituents. In a preferred embodiment R³=H especially in the group B=—CR³₂—.

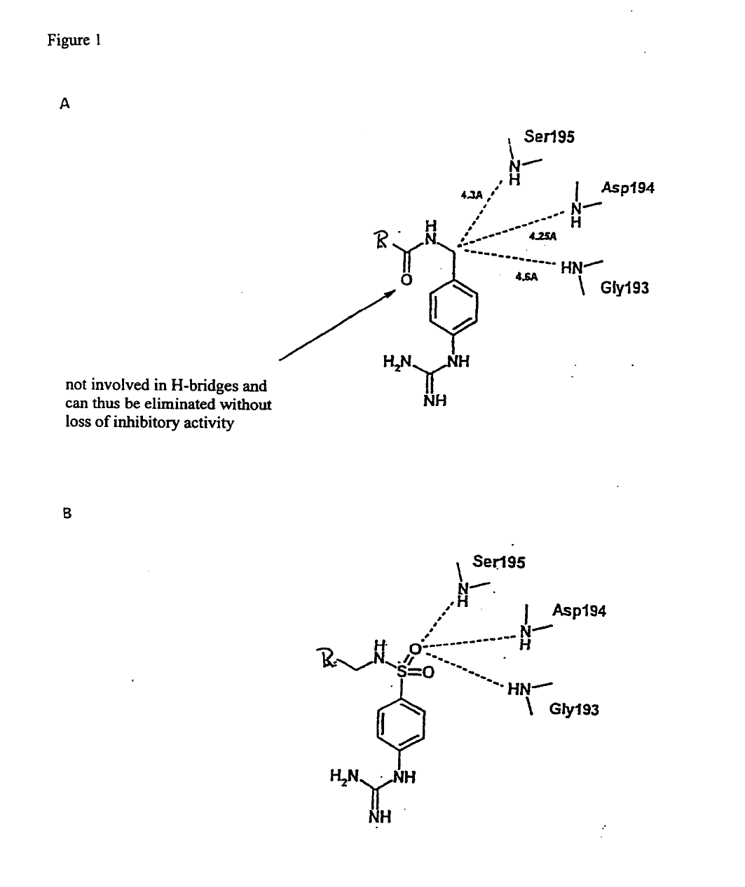

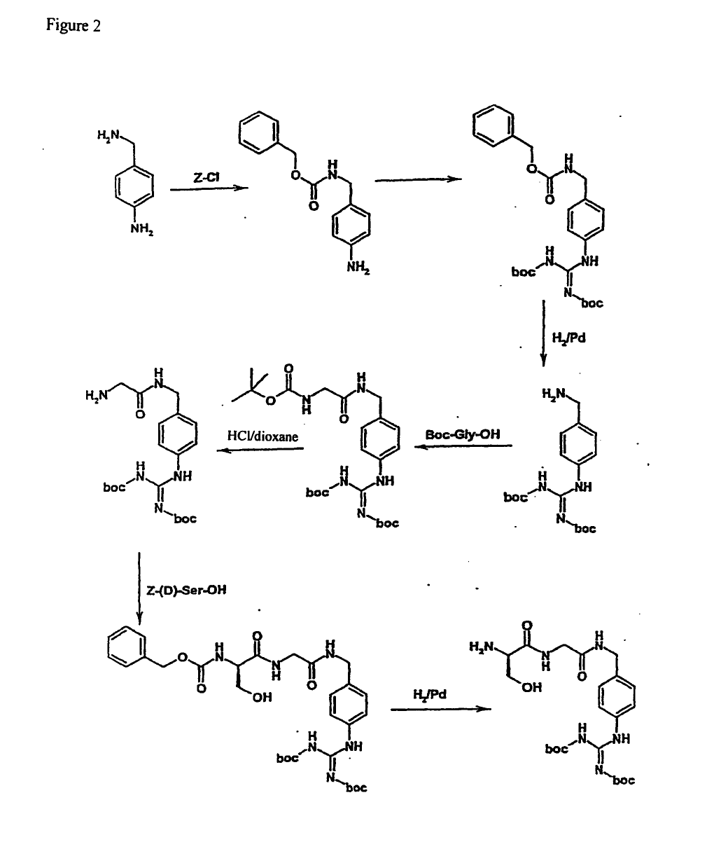

In a particularly preferred embodiment B represents the group —SO₂— so that they are sulfo compounds. This SO₂ group is isosteric to the CH₂ group. Replacing the CH₂ group by the isosteric SO₂ group enables the formation of additional H bridges to the NH groups of Gly 193, Asp 194 and Ser 195 of urokinase which further improves the inhibitory activity (cf. FIG. 1).

R¹³ represents a group of formula (IIa) or (IIb)

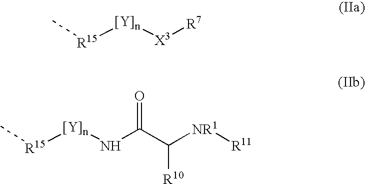

In formula (IIa) R¹⁵ denotes C=X² or CR³ where X² is NH, NR⁴, O or S and X³ is NH, NR⁴, O, S, CO, COO, CONH or CONR⁴. Y is C(R⁸), NH or NR³. R⁴ in turn denotes H or a branched or unbranched, optionally substituted alkyl, alkenyl or alkinyl residue. R⁷ denotes H or an optionally substituted alkyl, alkenyl, alkinyl, aryl or heteroaryl residue or —COR⁹ where R⁹ in turn represents H or a branched or unbranched, optionally substituted alkyl, alkenyl, alkinyl, aryl or/and heteroaryl residue.

R¹³ is preferably a group of formula (IIb).

In addition R¹³ preferably represents a group of formula (VIa) or (VIb) especially in compounds of formula (III).

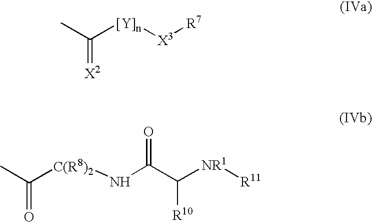

In formula (IVa) X² preferably denotes NH, NR⁴, O or S, in particular O and X³ represents NH, NR⁴, O, S, CO, COO, CONH or CONR⁴. Y represents C(R⁸)₂, NH or NR³. R⁴ in turn denotes H or a branched or unbranched, optionally substituted alkyl, alkenyl or alkinyl residue. R⁷ denotes H or an optionally substituted alkyl, alkenyl, alkinyl, aryl or heteroaryl residue or —COR⁹, where R⁹ in turn represents H or a branched or unbranched, optionally substituted alkyl, alkenyl, alkinyl, aryl or/and heteroaryl residue. In compounds of formula (III) R¹³ preferably represents a group of formula (IVb).

R⁸ is preferably hydrogen or (CH₂)ₘ—OH and particularly preferably H. R¹⁰ represents a residue (CRR¹)₂)ₙ—X⁵R⁵. In this case R¹ is particularly preferably hydrogen, X³ is particularly preferably oxygen, R⁵ is particularly preferably hydrogen and 0 is particularly preferably 1.

R¹¹ particularly preferably represents a sulfonyl residue —SO₂—R¹² where R¹² is preferably an aralkyl residue and in particular a benzyl residue. In a particularly preferred embodiment the benzyl residue is substituted at the meta and/or para position with halogen and most preferably with Cl. In another preferred embodiment R¹² is an adamantyl or camphor residue.

R¹⁵ particularly preferably represents a carbonyl residue —CO, amine residue —NR³— or/and alkyl residue —CR³₂, preferably —CR¹₂— and most preferably —CH₂—.

Compounds in which R¹⁵ represents CH₂ are characterized by a particularly simple synthesis. Since this position is not involved in the formation of hydrogen bridges with urokinase, a CH₂ group may be present instead of a carbonyl without being associated with a loss in inhibitory activity.

Compounds are also preferred which contain a non-natural amino acid as a building block especially for the residue R¹⁰. Furthermore aza compounds are preferred containing the group NH—NH in which for example Y=NH and n=1 (compounds of formula IIb).

Other particularly preferred compounds are bisulfonamides i.e. compounds which contain the element —SO₂—NH twice. Compounds are also preferred in which R¹³ represents a group of formula IIb and Y represents —C(R⁸)₂— where R⁸ once represents H and once represents a residue which contains an aromatic group and in particular —CH₂—CH₂—C₆H₅.

Compounds are also preferred in which X¹ denotes

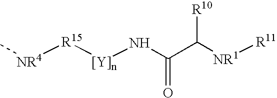

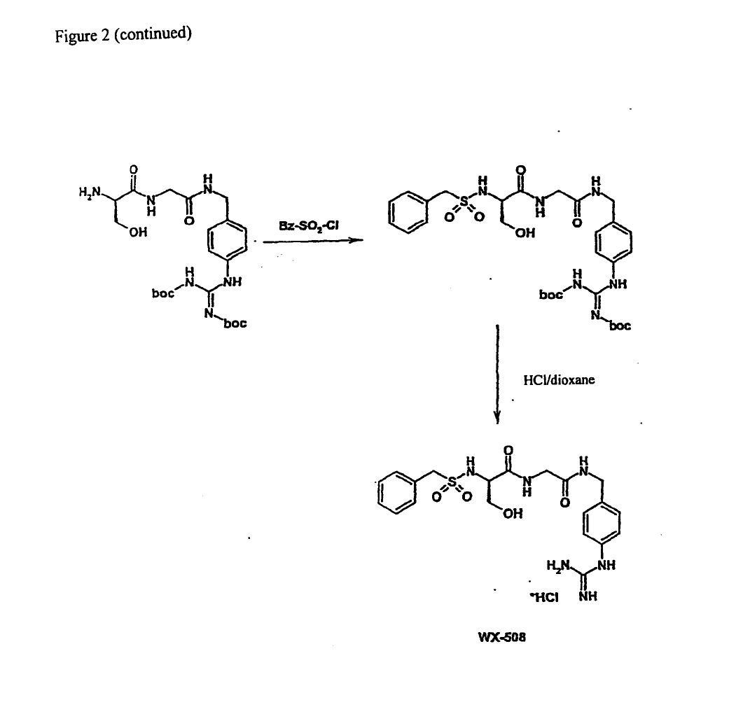

The following compounds are most preferred: N-[2-(4-guanidino-benzenesulfonyl-amino)-ethyl]-3-hydroxy-2-phenylmethanesulfonylaminopropionamide hydrochloride, Bz-SO₂-(D)-Ser-(Aza-Gly)-4-guanidino-benzylamide hydrochloride or N-(4-guanidino-benzyl)-2-(3-hydroxy-2-phenylmethane-sulfonylaminopropionyl-amino)-4-phenyl-butyramide hydrochloride and N-[(4-guanidino-benzylcarbamoyl)-methyl]-3-hydroxy-2-phenylmethanesulfonylaminopropionamide (see also FIG. 2: WX-508). Further preferred compounds are 3-nitrobenzyl-sulfonyl-(D)-Ser-Gly-(4-guanidinobenzyl)amide hydrochloride (WXC-316), 3-chlorobenzyl-sulfonyl-(D)-Ser-Gly-(4-guanidinobenzyl)amide hydrochloride (WXC-318), 4-chlorobenzyl-sulfonyl-(D)-Ser-Gly-(4-guanidinobenzyl)amide hydrochloride (WXC-340), benzylsulfonyl-(D)-Ser-Ala-(4-guanidinobenzyl)amide hydrochloride (WX-532), 4-chlorobenzylsulfonyl-(D)-Ser-N-Me-Ala-(4-guanidinobenzyl)amide (WX-582) or benzylsulfonyl-(D)-Ser-N-Me-Gly-(4-guanidinobenzyl)amide (WX-538).

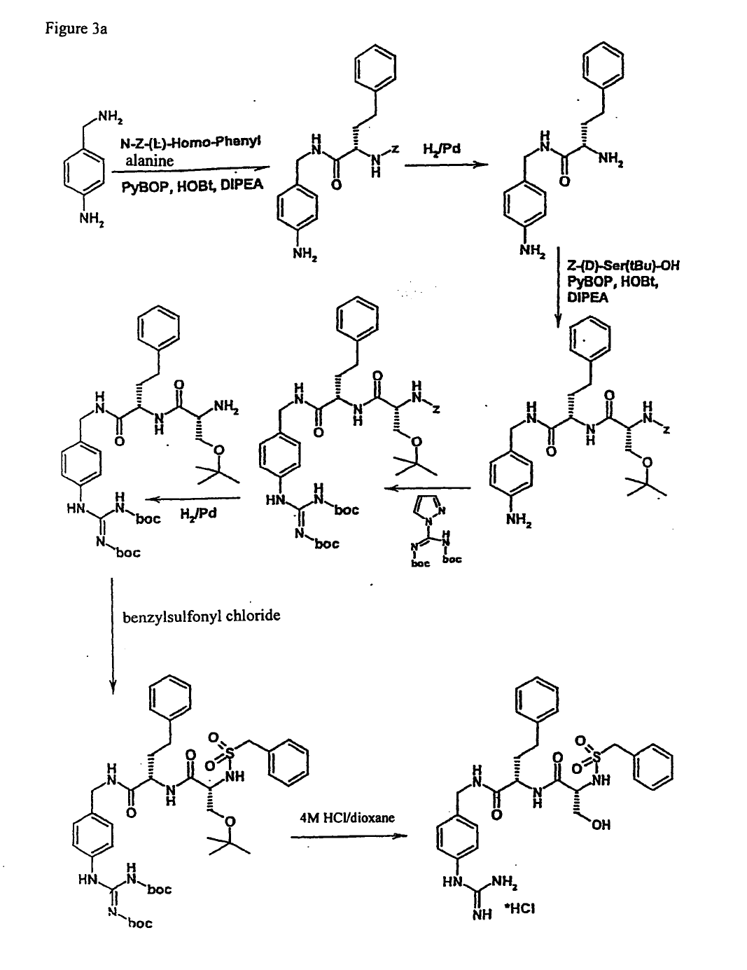

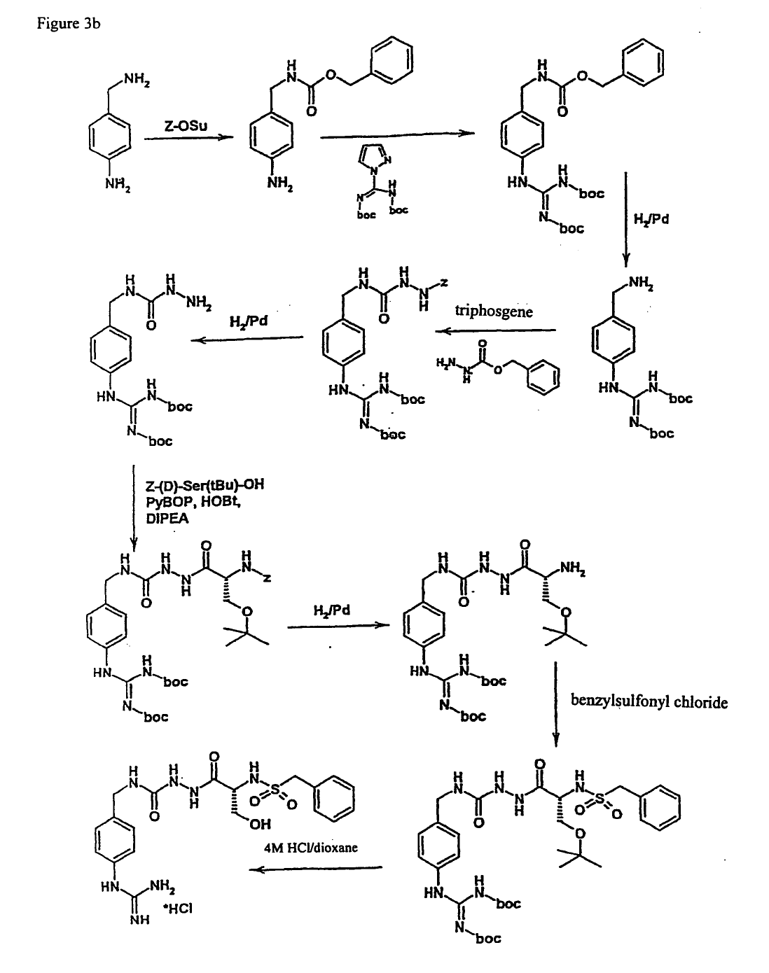

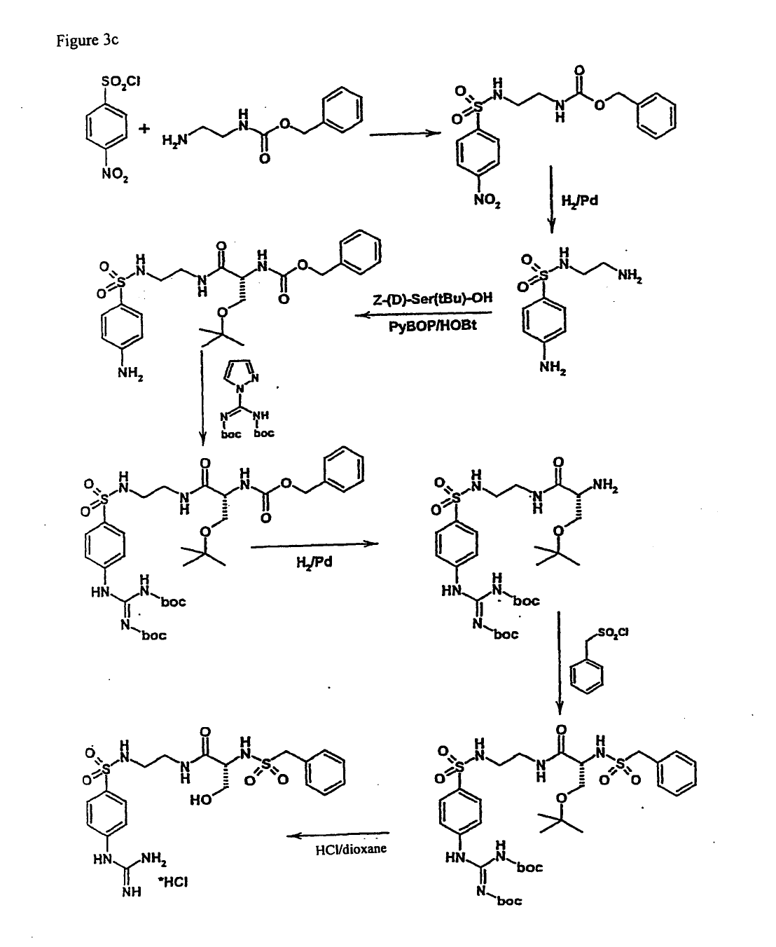

The compounds of the general formula (I) can for example be prepared as shown in the synthesis scheme in FIG. 2 and FIG. 3a, b and c with particularly preferred compounds as examples.

The urokinase inhibitors according to the invention can optionally be used together with suitable pharmaceutical auxiliary substances or vehicles to produce pharmaceutical preparations or for use in diagnostics. In this connection it is possible to administer them in combination with other active substances e.g. other urokinase inhibitors such as antibodies or/and peptides and also together with chemotherapeutic agents and cytostatic agents or/and cytostatic substances.

The pharmaceutical preparations can be administered to humans and animals topically, orally, rectally or parenterally e.g. intravenously, subcutaneously, intramuscularly, intraperitoneally, sublingually, nasally or/and by inhalation e.g. in the form of tablets, dragees, capsules, pellets, suppositories, solutions, emulsions, suspensions, liposomes, inhalation sprays or transdermal systems such as plasters.

The compounds according to the invention are suitable for combating diseases that are associated with a pathological overexpression of uPA or/and urokinase plasminogen activator receptor (uPAR). They are for example able to highly efficiently inhibit the growth or/and spread of malignant tumors as well as the formation of metastases by tumors. For this purpose the uPA inhibitors can optionally be used together with other tumor agents or with other types of treatment e.g. irradiation or surgical intervention. The inhibitors according to the invention are also effective for other uPA-associated or/and uPAR-associated diseases.

uPA inhibitors according to the invention are preferably characterized in that they have a Kᵢ value for uPA that is at least twice, preferably at least five-fold, particularly preferably at least ten-fold and up to 1000-fold lower than their Kᵢ for tPA, plasmin or/and thrombin. Furthermore it is remarkable that the compounds according to the invention only have a slight effect on blood coagulation since they have Kᵢ values that are too high for an effective inhibition of thrombin, plasmin and factor Xa.

The substances according to the invention of formula (I) or formula (III) can be used in combination with physiologically active substances e.g. with radiolabels or with cytotoxic agents e.g. chemotherapeutic agents such as cis-platinum or 5-fluoro-uracil or peptides. Furthermore, the substances can also be incorporated into membranes of carrier vesicles e.g. liposomes or/and be administered together with active substances e.g. cytotoxic agents such as doxorubicin that are enclosed in carrier vesicles.

The invention provides a method for urokinase inhibition in living organisms especially in humans by administration of an effective amount of at least one compound of the general formula (I). The dose of the compound is usually in the range of 0.01 to 100 mg/kg body weight per day. The duration of treatment depends on the severity of the disease and can range from a single administration to a treatment lasting several weeks or even many months which can optionally be repeated at intervals.

Finally the invention concerns new basic phenylalanine derivative of the general formulae (I) and (III).

The invention is further elucidated by the following examples and the attached figures.

FIG. 1 shows the interactions between inhibitors according to the invention in which B represents an SO₂ group and urokinase. It shows the hydrogen bridges that are formed between the SO₂ group and the NH groups of Gly 193, Asp 194 and Ser 195 in the backbone chain of urokinase.

FIG. 2 shows a synthesis scheme for preparing the particularly preferred compound N-[(4-guanidino-benzylcarbamoyl)-methyl]-3-hydroxy-2-phenyl-methanesulfonylaminopropionamide (WX-508). According to this general reaction scheme the compounds of formula (I) can be prepared starting from p-amino-benzylamine. In FIG. 2 Z denotes the protective group benzyloxycarbonyl and Boc denotes the protective group tert-butyloxycarbonyl.

FIG. 3 shows synthesis schemes for preparing the particularly preferred compounds WX-550 (FIG. 3a), WX-544 (FIG. 3b) and WX-568 (FIG. 3c). The compounds of formula (I) can be prepared according to this general reaction scheme starting from p-amino-benzylamine. In FIG. 3Z denotes the protective group benzyloxycarbonyl and Boc denotes the protective group tert.-butyloxycarbonyl.

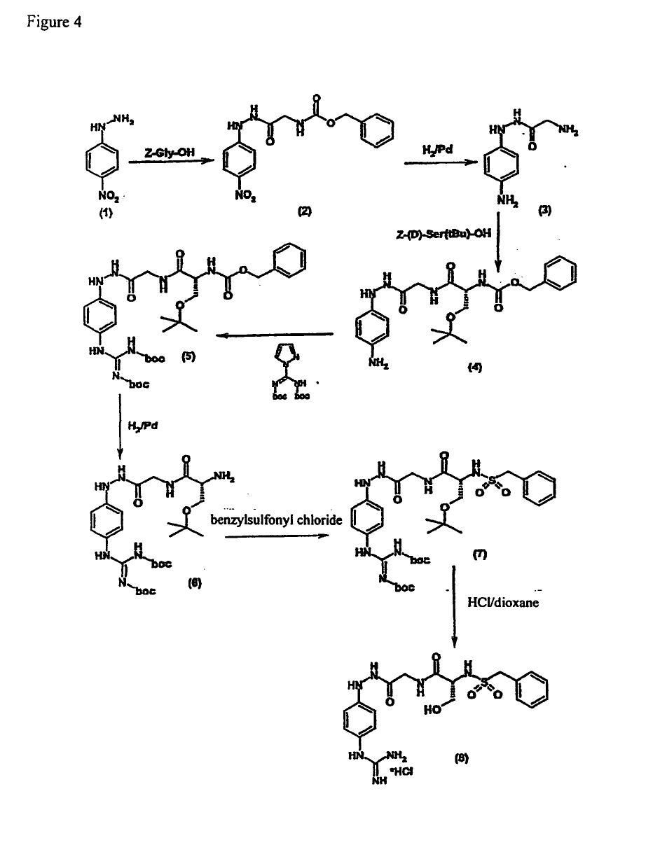

FIG. 4 shows a synthesis scheme for preparing the compound WX-600.

### EXAMPLES

The following abbreviations are used herein:

| | |
|---|---|
| HBTU | 2-(1H-benzotriazol-1-yl)-1,1,3,3- |
| | tetramethyluronium hexafluorophosphate |
| HOBt | N-hydroxybenzotriazole |
| PyBOP | benzotriazol-1-yl-oxy-tris- |
| | pyrrolidinophosphonium hexafluorophosphate |
| DCC | N,N′-dicyclohexylcarbodiimide |
| tBu | tert.-butyl |
| BOC | tert.-butyloxycarbonyl protective group |
| Z | benzyloxycarbonyl protective group |
| Z-OSu | N-(benzyloxycarbonyloxy) succinimide |
| TEA | triethylamine |
| DIPEA | diisopropylethylamine |
| Z-Gly-OSu | Nα-benzyloxycarbonylglycine-N- |
| | hydroxysuccinimide ester |
| TFA | trifluoroacetic acid |
| 4M | 4 molar |
| N-Z-N-Me-Gly | Nα-benzyloxycarbonyl-Nα-methyl-glycine |
| N-Z-N-Me-Ala | Nα-benzyloxycarbonyl-Nα-methyl-alanine |
| Z-HomoPhe-OH | Nα-benzyloxycarbonyl-homophenylalanine |
| Fmoc-(D)Dap(Z)-OH | Nα-fluorenyloxycarbonyl-N-benzyloxy- |
| | carbonyl-(D)-diamino-propionic acid |
| BOC-(D)-Ser(tBu)-OH | Nα-tert.-butyloxycarbonyl-O-tert.-butyl-(D)- |
| | serine |

### Example 1

Description of the Synthesis of N-[2-(4-guanidino-benzenesulfonylamino)-ethyl]-3-hydroxy-2-phenylmethanesulfonylamino-propionamide hydrochloride [WX-5681 (cf. FIG. 3c)

4-Nitrophenylsulfonyl chloride (1) is reacted with N-Z-diaminoethane hydrochloride (2) in an inert organic solvent (e.g. dichloromethane) with addition of an organic base (e.g. TEA, DIPEA) to form the compound 3. Catalytic hydrogenation of 3 on a Pd-active charcoal catalyst converts the nitrogroup into the corresponding amine and also cleaves the Z-protective group (4). The aminoethyl group of compound 4 can be converted to the corresponding amide 5 by using conventional coupling reagents (e.g. PyBOP, HBTU or DCC and HOBt) in peptide chemistry together with Z-(D)-Ser(tBu)-OH. Conversion into the completely protected intermediate 6 is carried out with a suitable guanidinylation reagent such as N,N′-Bis(tert.-butoxycarbonyl)-1H-pyrazole-1-carboxamidine. Subsequent cleavage of the Z-protective group by catalytic hydrogenation on a Pd-active charcoal catalyst (7) and reaction with benzyl-sulfonyl chloride in an inert organic solvent (e.g. dichloromethane) with addition of an organic base yields the completely protected product 8. The protective groups (BOC and t-butylether) are cleaved by dissolving compound 8 in acid (e.g. trifluoroacetic acid or 4 M HCl_{g} in dioxane) to obtain the corresponding salt of the target compound N-[2-(4-guanidinobenzenesulfonylamino)-ethyl]-3-hydroxy-2-phenylmethanesulfonylamino propionamide (9).

### Example 2

Description of the Synthesis of Bz-SO₂-(D)-Ser-(Aza-Gly)-4-guanidinobenzyl-amide hydrochloride [WX-5441 (cf. FIG. 3b)

The aminomethyl group of the educt 4-aminobenzylamine (10) is firstly provided with a suitable protective group by for example reacting 10 with benzyl chloroformate or Z-OSu to form compound 11. Reaction with a suitably protected guanidinylation reagent such as N,N′-Bis(tert-butoxycarbonyl)-1-H-pyrazole-1-carboxamidine yields compound 12 whose Z-protective group can be cleaved by catalytic hydrogenation on a Pd-active charcoal catalyst (13). The amino group that is released in this manner is reacted with triphosgene (a solid and thus less poisonous phosgene substitute) and is subsequently reacted in situ with benzyl carbazate while cooling and with addition of one equivalent of organic base to form compound 14 (Z-AzaGly-4-(N,N′-Bis-BOC-guanidinobenzyl)amide). The Z-protective group is cleaved by hydrogenation as described for compound 13 (15) and converted into the amide 16 with Z-(D)-Ser(tBu)-OH and common coupling reagents in peptide chemistry (e.g. PyBOP, HBTU or DCC and HOBt). The Z-protective group is again cleaved catalytically (17) and the resulting free amine is converted into the corresponding sulfonamide 18 in an inert organic solvent (e.g. dichloromethane) with addition of an organic base. The remaining protective groups are cleaved by reaction in acidic solution (e.g. in trifluoroacetic acid or in 4M HCl_{g}/dioxane) to form the corresponding salt of the target compound Bz-SO₂-(D)-Ser-(Aza-Gly)-4-guanidino-benzylamide (19).

### Example 3

Description of the Synthesis of N-(4-guanidino-benzyl)-2-(3-hydroxy-2-phenylmethane-sulfonylamino-propionylamino)-4-phenyl-butyramide hydrochloride [WX-550] (cf. FIG. 3a)

4-Aminobenzylamine (20) is reacted with Z-(L)-homophenylalanine and conventional coupling reagents in peptide chemistry (e.g. PyBOP, HBTU or DCC and HOBt) to form the amide 21. The Z-protective group is cleaved (22) by catalytic hydrogenation on a Pd-active charcoal catalyst and the free amino group is reacted with Z-(D)-Ser(tBu)-OH to form the amide 23 as described for 2.1. Reaction with a suitably protected guanidinylation reagent such as N,N′-bis(tert.-butoxycarbonyl)-1H-pyrazole-1-carboxamidine yields compound 24. The N-terminal Z-protective group is cleaved as described for 22 and the resulting compound 25 is converted into the sulfonamide 26 with benzylsulfonyl chloride. In the last step the remaining protective groups are cleaved in an acidic medium (e.g. in trifluoroacetic acid or in 4M HCl/dioxane) to form the corresponding salt of the target compound 27.

### Example 4

Description of the Synthesis of N-[(4-guanidino-benzylcarbamoyl]methyl]-3-hydroxy-2-phenyl-methanesulfonylamino-propionamide hydrochloride and benzylsulfonyl-(D)-Ser-Gly-(4-guanidinobenzyl)amide hydrochloride [WX-508] (cf. FIG. 2)

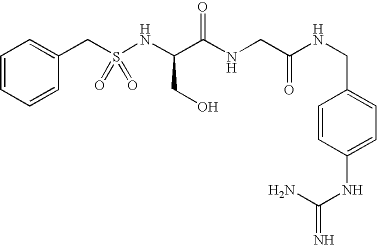

Commercially available 4-aminobenzylamine is reacted with Z-Gly-OSu in an inert solvent to form Z-Gly-(4-aminobenzyl)amide. Alternatively it is also possible to use Z-Gly-OH and conventional coupling reagents in peptide chemistry (e.g. DCC or HBTU and HOBt). After cleaving the Z-protective group by catalytic hydrogenation on a palladium/active charcoal catalyst, the free amino group is reacted with Z-(D)-Ser(tBu)-OH and with the aid of conventional coupling reagents in peptide chemistry (e.g. DCC or HBTU or HOBt) to form the corresponding Z-(D)Ser(tBu)-Gly-(4-aminobenzyl)amide. The BOC-protected guanidino function can for example be synthesized at this stage by reaction with N,N′-bis-BOC-1H-pyrazole-1-carboxamidine in a solvent (e.g. dichloromethane) that should be as unpolar and inert as possible. After a further catalytic cleavage of the Z-protective group, it is reacted with phenylmethanesulfonyl chloride to form the completely protected product BzSO₂-(D)-Ser(tBu)-Gly-(4-N,N′-bis-BOC-guanidinobenzyl)amide. In the last step in the synthesis the BOC-protective groups and the tert.-butyl ether group are cleaved by 4M HCl_{g} in dioxane to directly obtain the hydrochloride of N-[(4-guanidino-benzyl-carbamoyl)-methyl]-3-hydroxy-2-phenyl-methanesulfonylamino-propionamide.

Alternatively the protective groups can also be cleaved by TFA. The resulting TFA salt of the product is subsequently converted into the corresponding hydrochloride by ion exchange.

### Example 5

Synthesis of the Common Building Block H-(D)-Ser(tBu)-Gly-(4N,N′-bis-BOC-guanidino-benzyl)amide (1):

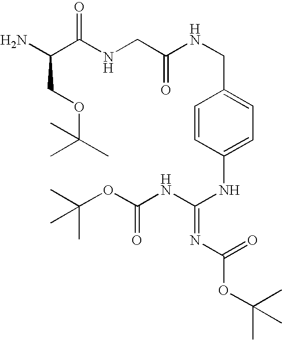

Commercially available 4-aminobenzylamine is reacted with Z-Gly-OSu in an inert solvent to form Z-Gly-(4-aminobenzyl)amide. [Alternatively it is also possible to use Z-Gly-OH and conventional coupling reagents in peptide chemistry (e.g. DCC or HBTU and HOBt)]. The BOC-protected guanidino function can for example be synthesized at this stage by reaction with N,N′-bis-BOC-1H-pyrazole-1-carboxamidine in a solvent (e.g. dichloromethane) that should be as unpolar and inert as possible. After cleavage of the Z-protective group by catalytic hydrogenation on a palladium/active charcoal catalyst, the free amino group is converted into the corresponding Z-(D)-Ser(tBu)-Gly-(4-N,N′-bis-BOC-guanidinobenzyl)amide with Z-(D)-Ser(tBu)-OH and with the aid of conventional coupling reagents in peptide chemistry (e.g. DCC or HBTU and HOBt). Cleavage of the Z-protective group by catalytic hydrogenation on a Pd-active charcoal catalyst yields H-(D)-Ser(tBu)-Gly-(4-N,N′-bis-BOC-guanidinobenzyl)amide as the building block (1) for the N-terminal derivatization reactions described in the following.

### Example 6

Synthesis of N-Terminal Sulfonamide Derivatives of the General Formula

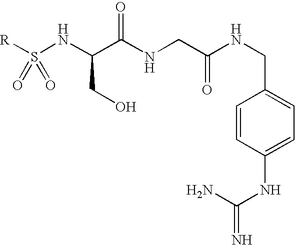

(Examples WXC-296, 298, 300, 302, 316, 318, 340)

In order to synthesize the N-terminal sulfonamide derivatives according to the invention, the building block 1 is reacted with one equivalent of the desired substituted sulfonyl chloride in the presence of a tertiary organic base (e.g. TEA or DIPEA) in an inert organic solvent (e.g. dichloromethane). Subsequently the protective groups of the fully protected product that is formed in this process are cleaved by 4M HCl_{g} in dioxane to obtain the hydrochloride of the desired compounds R—SO₂-(D)-Ser-Gly-(4-guanidinobenzyl)amide (R generally denotes an organic residue). Alternatively the protective groups can also be cleaved by TFA. The resulting TFA salt of the product is subsequently converted into the corresponding hydrochloride by ion exchange.

6.1. (−)-Camphor-10-sulfonyl-(D)-Ser-Gly-4-guanidinobenzyl)amide hydrochloride (WXC-296)

In order to synthesize the compound (−)-camphor-10-sulfonyl-(D)-Ser-Gly-(4-guandinobenzyl)amide hydrochloride (WXC-296) according to the invention, the building block 1 is reacted with one equivalent of (−)-camphor-10-sulfonyl chloride in the presence of a tertiary organic base (e.g. TEA or DIPEA) in an inert organic solvent (e.g. dichloromethane). Subsequently the protective groups of the fully protected product that is formed in this process are cleaved by 4M HCl_{g} in dioxane to obtain the hydrochloride of the target compound. Alternatively the protective groups can also be cleaved by TFA. The resulting TFA salt of the product is subsequently converted into the corresponding hydrochloride by ion exchange.

6.2. (+)-Camphor-10-sulfonyl-(D)-Ser-Gly-(4-guanidinobenzyl)amide hydrochloride (WXC-298)

(+)-Camphor-10-sulfonyl-(D)-Ser-Gly-(4-guandinobenzyl)amide hydrochloride (WXC-298) is synthesized as described under 6.1 but using (+)-camphor-10-sulfonyl chloride.

6.3. n-Butyl-sulfonyl-(D)-Ser-Gly-(4-guanidinobenzyl)amide hydrochloride (WXC-300)

n-Butyl-sulfonyl-(D)-Ser-Gly-(4-guanidinobenzyl)amide hydrochloride (WXC-300) is synthesized as described under 6.1 but using butylsulfonyl chloride.

6.4 n-Octyl-sulfonyl-(D)-Ser-Gly-(4-guanidinobenzyl)amide hydrochloride (WXC-302)

n-Octyl-sulfonyl-(D)-Ser-Gly-(4-guanidinobenzyl)amide hydrochloride (WXC-302) is synthesized as described under 6.1 but using n-octylsulfonyl chloride.

6.5 3-Nitrobenzyl-sulfonyl-(D)-Ser-Gly-(4-guanidinobenzyl)amide hydrochloride (WXC-316)

3-Nitrobenzyl-sulfonyl-(D)-Ser-Gly-(4-guanidinobenzyl)amide hydrochloride (WXC-316) is synthesized as described under 6.1 but using 3-nitrobenzyl-sulfonyl chloride.

6.6 3-Chlorobenzyl-sulfonyl-(D)-Ser-Gly-(4-guanidinobenzyl)amide hydrochloride (WXC-318)

3-Chlorobenzyl-sulfonyl-(D)-Ser-Gly-(4-guanidinobenzyl)amide hydrochloride (WXC-318) is synthesized as described under 6.1 but using 3-chlorobenzyl-sulfonyl chloride.

6.7 4-Chlorobenzyl-sulfonyl-(D)-Ser-Gly-(4-guanidinobenzyl)amide hydrochloride (WXC-340)

4-Chlorobenzy]-sulfonyl-(D)-Ser-Gly-(4-guanidinobenzyl)amide hydrochloride (WXC-340) is synthesized as described under 6.1 but using 4-chlorobenzyl-sulfonyl chloride.

### Example 7

Synthesis of N-Terminal Urea Derivatives of the General Formula

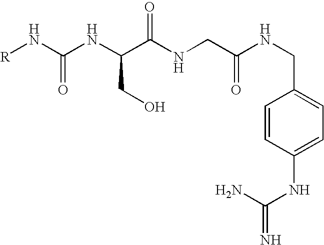

(Examples WXC-202, 304, 306, 308, 310, 312, 314, 320, 322, 324, 326, 328, 330, 332, 334, 336, 338, 342)

In order to synthesize the N-terminal urea derivatives according to the invention, the building block 1 is reacted with one equivalent of the desired substituted isocyanate in an inert organic solvent (e.g. dichloromethane). Subsequently the protective groups of the fully protected product that is formed in this process are cleaved by 4M HCl_{g} in dioxane to obtain the hydrochloride of the desired compounds N-[(4-guanidino-benzylcarbamoyl)methyl]-3-hydroxy-2-(3-R-(ureido)-propionamide (R generally denotes an organic residue). Alternatively the protective groups can also be cleaved by TFA. The resulting TFA salt of the product is subsequently converted into the corresponding hydrochloride by ion exchange.

7.1 3-Chlorophenyl-aminocarbonyl-(D)-Ser-Gly-(4-guanidinobenzyl)amide hydrochloride (WXC-292)

In order to synthesize the compound 3-chlorophenyl-aminocarbonyl-(D)-Ser-Gly-(4-guanidinobenzyl)amide hydrochloride (WXC-292) according to the invention, the building block 1 is reacted with one equivalent of 3-chlorophenyl isocyanate in an inert organic solvent (e.g. dichloromethane). Subsequently the protective groups of the fully protected product that is formed in this process are cleaved by 4M HCl_{g} in dioxane to obtain the hydrochloride of the desired product. Alternatively the protective groups can also be cleaved by TFA. The resulting TFA salt of the product is subsequently converted into the corresponding hydrochloride by ion exchange.

7.2 2-Chlorophenyl-aminocarbonyl-(D)-Ser-Gly-(4-guanidinobenzyl)amide hydrochloride (WXC-304)

2-Chlorophenyl-aminocarbonyl-(D)-Ser-Gly-(4-guanidinobenzyl)amide hydrochloride (WXC-304) is synthesized as described under 7.1 but using 2-chlorophenyl isocyanate.

7.3 4-Methoxyphenyl-aminocarbonyl-(D)-Ser-Gly-(4-guanidinobenzyl)amide hydrochloride (WXC-306)

4-Methoxyphenyl-aminocarbonyl-(D)-Ser-Gly-(4-guanidinobenzyl)amide hydrochloride (WXC-306) is synthesized as described under 7.1 but using 4-methoxyphenyl isocyanate.

7.4 3,4,5-Tri-methoxyphenyl-aminocarbonyl-(D)-Ser-Gly-(4-guanidino-benzyl)amide hydrochloride (WXC-308)

3,4,5-Tri-methoxyphenyl-aminocarbonyl-(D)-Ser-Gly-(4-guanidinobenzyl)amide hydrochloride (WXC-308) is synthesized as described under 7.1 but using 3,4,5-tri-methoxyphenyl isocyanate.

7.5 4-Phenoxyphenyl-aminocarbonyl-(D)-Ser-Gly-(4-guanidinobenzyl)amide hydrochloride (WXC-310)

4-Phenoxyphenyl-aminocarbonyl-(D)-Ser-Gly-(4-guanidinobenzyl)amide hydrochloride (WXC-310) is synthesized as described under 7.1 but using 4-phenoxyphenyl isocyanate.

7.6 3-Ethoxycarbonylphenyl-aminocarbonyl-(D)-Ser-Gly-(4-guanidino-benzyl)amide hydrochloride (WXC-312)

3-Ethoxycarbonylphenyl-aminocarbonyl-(D)-Ser-Gly-(4-guanidinobenzyl)amide hydrochloride (WXC-312) is synthesized as described under 7.1 but using ethyl-3-isocyanato benzoate.

7.7 3-Acetylphenyl-aminocarbonyl-(D)-Ser-Gly-(4-guanidinobenzyl)amide hydrochloride (WXC-314)

3-Acetylphenyl-aminocarbonyl-(D)-Ser-Gly-(4-guanidinobenzyl)amide hydrochloride (WXC-314) is synthesized as described under 7.1 but using 3-acetylphenyl isocyanate.

7.8 1-Adamantyl-aminocarbonyl-(D)-Ser-Gly(4-guanidinobenzyl)amide hydrochloride (WXC-320)

1-Adamantyl-aminocarbonyl-(D)-Ser-Gly-(4-guanidinobenzyl)amide hydrochloride (WXC-320) is synthesized as described under 7.1 but using 1-adamantyl isocyanate.

7.9 2-Bromophenyl-aminocarbonyl-(D)-Ser-Gly-(4-guanidinobenzyl)amide hydrochloride (WXC-322)

2-Bromophenyl-aminocarbonyl-(D)-Ser-Gly-(4-guanidinobenzyl)amide hydrochloride (WXC-322) is synthesized as described under 7.1 but using 2-bromophenyl isocyanate.

7.10 3-Carboxyphenyl-aminocarbonyl-(D)-Ser-Gly-(4-guanidinobenzyl)amide hydrochloride (WXC-324)

3-Carboxyphenyl-aminocarbonyl-(D)-Ser-Gly-(4-guanidinobenzyl)amide hydrochloride (WXC-324) is synthesized as described under 7.1 but using tert.-butyl-3-isocyanatobenzoate. The tert.-butyl protective group is cleaved together with the other acid-labile protective groups by treatment with 4M HCl_{g} in dioxane.

7.11 2,3-Dihydro-1,4-benzodioxin-6-yl-aminocarbonyl-(D)-Ser-Gly-4-guanidinobenzyl)amide hydrochloride (WXC-326)

2,3-Dihydro-1,4-benzodioxin-6-yl-aminocarbonyl-(D)-Ser-Gly-(4-guanidinobenzyl)amide hydrochloride (WXC-326) is synthesized as described under 7.1 but using 2,3-dihydro-1,4-benzodioxin-6-yl isocyanate.

7.12 1-Naphthyl-aminocarbonyl-(D)-Ser-Gly-(4-guanidinobenzyl)amide hydrochloride (WXC-328)

1-Naphthyl-aminocarbonyl-(D)-Ser-Gly-(4-guanidinobenzyl)amide hydrochloride (WXC-328) is synthesized as described under 7.1 but using 1-naphthyl isocyanate.

7.13 4-Acetylphenyl-aminocarbonyl-(D)-Ser-Gly-(4-guanidinobenzyl)amide hydrochloride (WXC-330)

4-Acetylphenyl-aminocarbonyl-(D)-Ser-Gly-(4-guanidinobenzyl)amide hydrochloride (WXC-328) is synthesized as described under 7.1 but using 4-acetylphenyl isocyanate.

7.14 3,4-Methylenedioxyphenyl-aminocarbonyl-(D)-Ser-Gly-(4-guanidinobenzyl)amide hydrochloride (WXC-332)

3,4-Methylenedioxyphenyl-aminocarbonyl-(D)-Ser-Gly-(4-guanidinobenzyl)amide hydrochloride (WXC-332) is synthesized as described under 7.1 but using 3,4-methylenedioxyphenyl isocyanate.

7.15 2,3-Dichlorophenyl-aminocarbonyl-(D)-Ser-Gly-(4-guanidinobenzyl)-amide hydrochloride (WXC-334)

2,3-Dichlorophenyl-aminocarbonyl-(D)-Ser-Gly-(4-guanidinobenzyl)amide hydrochloride (WXC-334) is synthesized as described under 7.1 but using 2,3-dichlorophenyl isocyanate.

7.16 4-Ethyloxycarbonylphenyl-aminocarbonyl-(D)-Ser-Gly-(4-guanidinobenzyl)amide hydrochloride (WXC-336)

4-Ethyloxycarbonylphenyl-aminocarbonyl-(D)-Ser-Gly-(4-guanidinobenzyl)amide hydrochloride (WXC-336) is synthesized as described under 7.1 but using 4-ethyloxycarbonylphenyl isocyanate.

7.17 2,4-Dibromophenyl-aminocarbonyl-(D)-Ser-Gly-4-guanidinobenzyl)-amide hydrochloride (WXC-338)

2,4-Dibromophenyl-aminocarbonyl-(D)-Ser-Gly-(4-guanidinobenzyl)amide hydrochloride (WXC-338) is synthesized as described under 7.1 but using 2,4-dibromophenyl isocyanate.

7.18 4-Nitrophenyl-aminocarbonyl-(D)-Ser-Gly-(4-guanidinobenzyl)amide hydrochloride (WXC-342)

4-Nitrophenyl-aminocarbonyl-(D)-Ser-Gly-(4-guanidinobenzyl)amide hydrochloride (WXC-342) is synthesized as described under 7.1 but using 4-nitrophenyl isocyanate.

### Example 8

Synthesis of N-Terminal Amide Derivatives of the General Formula

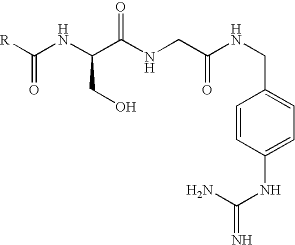

(Example WX-571)

In order to synthesize N-terminal amide derivatives according to the invention the building block 1 is reacted with one equivalent of the desired substituted acyl chloride in the presence of a tertiary organic base (e.g. TEA or DIPEA) in an inert organic solvent (e.g. dichloromethane). Alternatively carboxylic acids can also be converted into the desired amide with conventional coupling reagents in peptide chemistry (e.g. DCC or HBTU or HOBt). Other forms of carboxylic acid activation e.g. as pentafluorophenyl esters, hydroxysuccinimide esters or as anhydrides are also possible. Subsequently the protective groups of the fully protected product formed in this process are cleaved by 4M HCl_{g} in dioxane to obtain the hydrochloride of the desired compounds N-[(4-guanidinobenzylcarbamoyl)-methyl]-3-hydroxy-2-R-acylamino-propionamide (R generally denotes an organic residue). Alternatively the protective groups can also be cleaved by TFA. The resulting TFA salt of the product is subsequently converted into the corresponding hydrochloride by ion exchange.

8.1 3,4-Dihydroxyphenyl-acetyl-(D)-Ser-Gly-(4-guanidinobenzyl)amide hydrochloride (WX-571)

In order to synthesize the compound 3,4-dihydroxyphenyl-acetyl-(D)-Ser-Gly-(4-guanidino-benzyl)amide hydrochloride (WX-571) according to the invention the building block 1 is reacted with one equivalent of 3,4-dihydroxyphenylacetic acid and 1.1 equivalents of a conventional coupling reagent in peptide chemistry (e.g. DCC or HBTU or HOBt) in the presence of a tertiary organic phase (e.g. TEA or DIPEA) in an inert organic solvent (e.g. dichloromethane). Subsequently the protective groups of the fully protected product formed in this process are cleaved by 4M HCl_{g} in dioxane to obtain the hydrochloride of the desired compounds. Alternatively the protective groups can also be cleaved by TFA. The resulting TFA salt of the product is subsequently converted into the corresponding hydrochloride by ion exchange.

### Example 9

Synthesis of BzSO₂-(D)-Ser-AzaGly-(4-guanidinobenzyl)amide (WX-544)

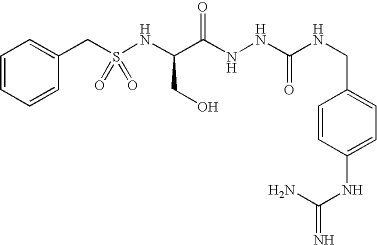

In order to synthesize the compound BzSO₂-(D)-Ser-AzaGly-(4-guanidinobenzyl)amide according to the invention, the aminomethyl group of 4-aminobenzylamine is firstly converted with Z-OSu into N-Z-(4-aminobenzyl)amine. The BOC-protected guanidino group can for example be synthesized at this stage by reaction with N,N′-bis-BOC-1H-pyrazole-1-carboxamidine in a solvent (e.g. dichloromethane) which should be as unpolar and inert as possible. After cleavage of the Z-protective group by catalytic hydrogenation on a palladium/active charcoal catalyst, the free amino group is firstly reacted in an inert solvent (e.g. dichloromethane) with ⅓ equivalent triphosgene and one equivalent TEA while cooling on ice, and after the reaction is completed with benzyl carbazate to form Z-AzaGly-(4-N,N′-bis-BOC-guanidinobenzyl)amide. After cleavage of the Z-protective group by catalytic hydrogenation on a palladium/active charcoal catalyst, the free amino group is converted with Z-(D)-Ser(tBu)-OH into the corresponding Z-(D)-Ser(tBu)-AzaGly-(4-N,N′-bis-BOC-guanidinobenzyl)amide with the aid of conventional coupling reagents in peptide chemistry (e.g. DCC or HBTU and HOBt). After another catalytic cleavage of the Z-protective group it is reacted with phenylmethanesulfonyl chloride to form the fully protected product BzSO₂-(D)-Ser(tBu)-AzaGly-4(N,N′-bis-BOC-guanidinobenzyl)amide. In the last step in the synthesis the BOC-protective groups and the tert.-butyl ether group is cleaved by 4M HCl_{g} in dioxane to directly obtain the hydrochloride of BzSO₂-(D)-Ser-AzaGly-4-(guanidinobenzyl)amide. Alternatively the protective groups can also be cleaved by TFA. The resulting TFA salt of the product is subsequently converted into the corresponding hydrochloride by ion exchange.

### Example 10

Synthesis of Compounds According to the Invention of the BzSO₂-(D)-Ser-Aaa-(4-guanidinobenzyl)amide type (Aaa Generally Denotes a Natural or Non-Natural α-amino Acid in the R or S Configuration or a Racemate Thereof)

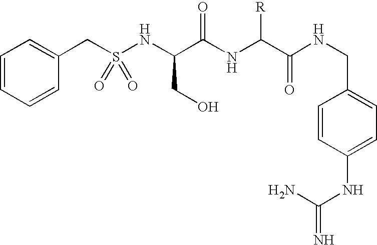

(Example WX-550)

Commercially available 4-aminobenzylamine is reacted in an inert solvent with the desired Z-protected amino acid (Z-Aaa) and conventional coupling reagents in peptide chemistry (e.g. DCC or HBTU and HOBt) to form Z-Aaa-(4-aminobenzyl)-amide. Reactive groups that may be present in the side chain of the amino acid Aaa should if possible be provided with an acid labile protective group (e.g. BOC, trityl, tert.-butyl ester or tert.-butyl ether) in this process. After cleaving the Z-protective group by catalytic hydrogenation on a palladium/active charcoal catalyst, the free amino group is converted with Z-(D)-Ser(tBu)-OH with the aid of conventional coupling reagents in peptide chemistry (e.g. DCC or HBTU and HOBt) into the corresponding Z-(D)-Ser(tBu)-Aaa-(4-aminobenzyl)amide. The BOC-protected guanidino group can be synthesized at this stage by reaction with N,N′-bis-BOC-1H-pyrazole-1-carboxamidine in a solvent (e.g. dichloromethane) which should be as unpolar and inert as possible. After another catalytic cleavage of the Z-protective group it is reacted with phenylmethanesulfonyl chloride to form the fully protected product BzSO₂-(D)-Ser(tBu)-Gly-4-(N,N′-bis-BOC-guanidinobenzyl)amide. In the last step of the synthesis the BOC-protective groups and the tert.-butylether group are cleaved by 4M HCl_{g} in dioxane to directly obtain the hydrochloride of BzSO₂-(D)-Ser-Aaa-(4-guanidinobenzyl)amide. Alternatively the protective groups can also be cleaved by TFA. The resulting TFA salt of the product is subsequently converted into the corresponding hydrochloride by ion exchange.

10.1 Benzylsulfonyl-(D)-Ser-HomoPhe-(4-guanidinobenzyl)amide hydrochloride (WX-550)

Commercially available 4-aminobenzylamine is reacted in an inert solvent (e.g. dichloromethane) with Z-HomoPhe-OH and conventional coupling reagents in peptide chemistry (e.g. DCC or HBTU and HOBt) to form Z-HomoPhe-(4-aminobenzyl)-amide.

After cleaving the Z-protective group by catalytic hydrogenation on a palladium/active charcoal catalyst, the free amino group is converted with Z-(D)-Ser(tBu)-OH with the aid of conventional coupling reagents in peptide chemistry (e.g. DCC or HBTU and HOBt) into the corresponding Z-(D)-Ser(tBu)-HomoPhe-4-aminobenzyl)amide. The BOC-protected guanidino group can be synthesized at this stage by reaction with N,N′-bis-BOC-1H-pyrazole-1-carboxamidine in a solvent (e.g. dichloromethane) which should be as unpolar and inert as possible. After another catalytic cleavage of the Z-protective group it is reacted with phenylmethanesulfonyl chloride to form the fully protected product BzSO₂-(D)-Ser(tBu)-HomoPhe-4-(N,N′-bis-BOC-guanidinobenzyl)amide. In the last step of the synthesis the BOC-protective groups and the tert.-butylether group are cleaved by 4M HCl_{g} in dioxane to directly obtain the hydrochloride of BzSO₂-(D)-Ser-HomoPhe-(4-guanidinobenzyl)amide.

Alternatively the protective groups can also be cleaved by TFA. The resulting TFA salt of the product is subsequently converted into the corresponding hydrochloride by ion exchange.

10.2 Benzylsulfonyl-(D)-Ser-Ala-(4-guanidinobenzyl)amide hydrochloride (WX-532)

Benzylsulfonyl-(D)-Ser-Ala-(4-guanidinobenzyl)amide hydrochloride (WX-532) is synthesized as described in 10.1 but using Z-Ala-OH instead of Z-HomoPhe-OH.

### Example 11

11.1 Synthesis of the Inventive Compound 2-(4-chlorophenylmethanesulfonylamino)-N-[1-(4-guanidino-benzylacarbamoyl)-ethyl]-3-hydroxy-N-methyl-propionamide and 4-clorobenzylsulfonyl-(D)-Ser-N-Me-Ala-(4-guanidinobenzyl)amide (WX-582)

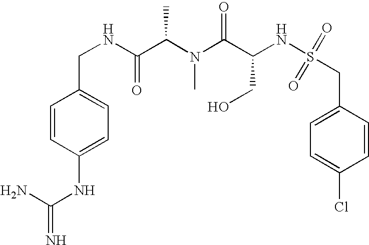

In order to synthesize the compound according to the invention, N-Z-N-methyl-alanine is converted with 4-aminobenzylamine into N-Z-N-methyl-alanine-(4-aminobenzyl)amide using conventional coupling reagents in peptide chemistry (e.g. DCC or HBTU or HOBt). After cleaving the Z-protective group by catalytic hydrogenation on a palladium/active charcoal catalyst, the free amino group is converted with Z-(D)-Ser(tBu)-OH with the aid of conventional coupling reagents in peptide chemistry (e.g. DCC or HBTU and HOBt) into the corresponding Z-(D)-Ser(tBu)-N-methyl-Ala-(4-aminobenzyl)amide. The BOC-protected guanidino group can be synthesized at this stage by reaction with N,N′-bis-BOC-1H-pyrazole-1-carboxamidine in a solvent (e.g. dichloromethane) which should be as unpolar and inert as possible. After another catalytic cleavage of the Z-protective group it is reacted with 4-chlorophenylmethanesulfonyl chloride to form the fully protected product 4Cl-BzSO₂-(D)-Ser(tBu)-N-methyl-Ala-4-(N,N′-bis-BOC-guanidinobenzyl)amide.

In the last step of the synthesis the BOC-protective groups and the tert.-butylether group are cleaved by 4M HCl_{g} in dioxane to directly obtain the hydrochloride of 2-(4-chlorophenyl-methanesulfonylamino)-N-[1-(4-guanidinobenzylcarbamoyl)-ethyl]-3-hydroxy-N-methyl-propionamide. Alternatively the protective groups can also be cleaved by TFA. The resulting TFA salt of the product is subsequently converted into the corresponding hydrochloride by ion exchange.

11.2 Synthesis of benzylsulfonyl-(D)-Ser-N-Me-Gly-(4-guanidinobenzyl)amide (WX-538)

The inventive compound benzylsulfonyl-(D)-Ser-N-Me-Gly-(4-guanidinobenzyl)amide (WX-538) is synthesized analogously to 11.1 but using N-Z-N-Me-Gly-OH instead of N-Z-N-methyl-alanine and benzylsulfonyl chloride instead of 4-chlorobenzylsulfonyl chloride.

### Example 12

### Synthesis of N-[N′-guanidino-phenyl)-hydrazinocarbonyl-methyl]-3-hydroxy-2-phenyl-methanesulfonylamino-propionamide hydrochloride (WX-600) (cf. FIG. 4)

4-Nitrophenylhydrazine (1) is reacted with Z-Gly-OH and a conventional coupling reagent in peptide chemistry (e.g. PyBOP, HBTU or DCC and HOBt) in an inert organic solvent (e.g. dichloromethane of DMF) to form compound 2. Catalytic hydrogenation on a palladium/active charcoal catalyst cleaves the Z-protective group and also reduces the nitro group to the corresponding amino group to obtain aminoacetic acid-N′-(4-amino-phenyl)hydrazine (3). The aliphatic amino group can be converted into the amide 4 by conventional coupling reagents in peptide chemistry (e.g. PyBOP, HBTU or DCC and HOBt) and Z-(D)-Ser-(tBu)-OH in an inert organic solvent (e.g. dichloromethane or DMF). The bis-BOC-protected guanidino group (5) is synthesized by reaction with N,N-bis(tert.-butoxycarbonyl)-1H-pyrazole-1-carboxamidine in an inert organic solvent (e.g. dichloromethane). The Z-protective group is cleaved by a further catalytic hydrogenation on a palladium/active charcoal catalyst (6). The subsequent reaction with 1 equivalent of benzylsulfonyl chloride in dichloromethane yields the fully protected product 7 which is deprotected by reaction with 4M HCl_{g} in dioxane to form the hydrochloride of the desired product N-[N′-(4-guanidino-phenyl)-hydrazinocarbonylmethyl]-3-hydroxy-2-phenylmethanesulfonyl-amino propionamide (8).

### Example 13

Synthesis of Derivatives Having the General Formula

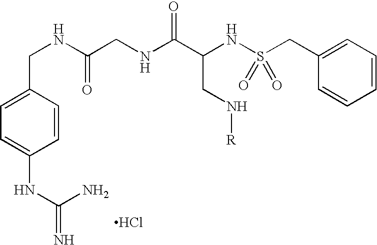

13.1. Synthesis of benzylsulfonyl-(D)-Dap(Z)-Gly-(4-guanidinobezyl-amide hydrochloride (WXM-5)

Commercially available 4-aminobenzylamine is reacted in an inert solvent with Z-Gly-OSu to form Z-Gly-(4-aminobenzyl)amide. (Alternatively Z-Gly-OH and conventional coupling reagents in peptide chemistry (e.g. DCC or HBTU and HOBt) can be used. After cleaving the Z-protective group by catalytic hydrogenation on a palladium/active charcoal catalyst, the free amino group is converted with Fmoc-(D)-Dap(Z)-OH with the aid of conventional coupling reagents in peptide chemistry (e.g. DCC or HBTU and HOBt) into the corresponding Fmoc-(D)-Dap(Z)-Gly-(4-aminobenzyl)amide. The BOC-protected guanidino group can be synthesized at this stage by reaction with N,N′-bis-BOC-1H-pyrazole-1-carboxamidine in a solvent (e.g. dichloromethane) which should be as unpolar and inert as possible. After cleavage of the Fmoc protective group by a secondary organic base (e.g. diethylamine or piperidine), it is reacted with phenylmethanesulfonyl chloride to form the protected product BzSO₂-(D)-Dap(Z)-Gly-(4-N,N′-bis-BOC-guanidinobenzyl)amide. In the last step of the synthesis the BOC-protective groups are cleaved by 4M HCl_{g} in dioxane to directly obtain the hydrochloride of benzylsulfonyl-(D)-Dap(Z)-Gly-(4-guanidinobenzyl)amide hydrochloride (WXM-5). Alternatively the protective groups can also be cleaved by TFA. The resulting TFA salt of the product is subsequently converted into the corresponding hydrochloride by ion exchange.

13.2 Synthesis of benzylsulfonyl-(D)-Dap-Gly-(4-guanidinobenzyl)amide his-hydrochloride (WXM-6)

Benzylsulfonyl-(D)-Dap-Gly-(4-guanidinobenzyl)amide bis-hydrochloride (WXM-6) can be synthesized by catalytic hydrogenation of WXM-5 (see 13.1) on a palladium/active charcoal catalyst in methanol which contains 2 molar equivalents of 1M HCl.

### Example 14

In Vitro Inhibition of Urokinase and Plasmin

In order to determine the inhibitory activity, 200 μl Tris buffer (0.05 mol/l containing the inhibitor, 0.154 mol/l NaCl, pH 8.0), 25 μl substrate (Pefachrome uPA, Pefachrome PL or Pefabloc TH in H₂O; Pentapharm Ltd, Basle, Switzerland) and 50 μl urokinase (Calbiochem-Novabiochem GmbH, Bad Soden, Germany) or another appropriate protease e.g. plasmin, thrombin or FXa are incubated for 10 minutes at 30° C. After ca. 30 minutes and 30 cycles the absorption is determined at 405 nm by means of a microplate reader (Mediators PHL, Aureon Biosystems, Vienna, Austria). The Kᵢ values were determined according to Dixon by linear regression using a computer program. The Kᵢ values are the mean of at least three determinations, the standard deviation was below 25%.

| | | Ki [μM] | Ki [μM] | Ki ]μM] | Ki [μM] |
|---|---|---|---|---|---|
| Formula | name | Plasmin | Thrombin | FXa | uPA |
| 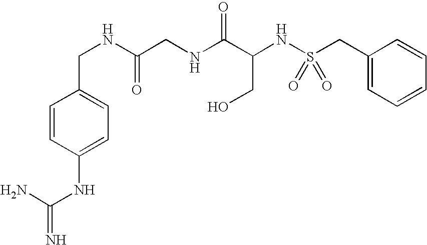 | WX-508 | >200 | >350 | | 0.034 |
| 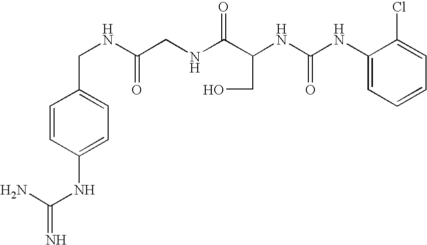 | WX-C304 | | | | no inh. *) |
| 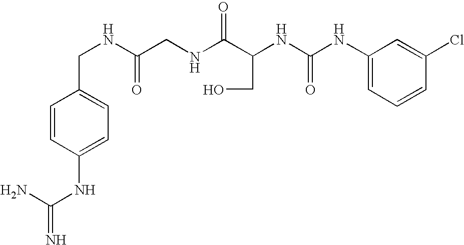 | WX-C292 | | | | no inh. *) |
| 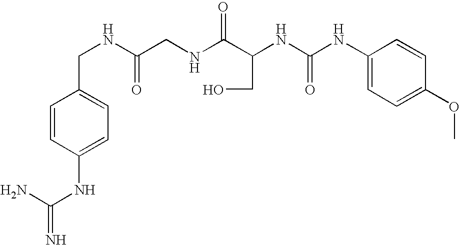 | WX-C306 | | | | 4.1 |
| 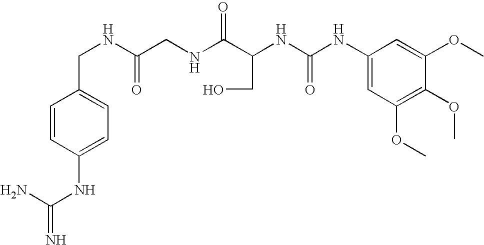 | WX-C308 | | | | >20 |
| 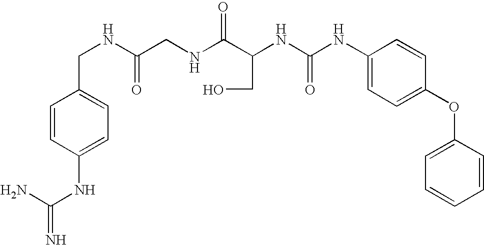 | WX-C310 | | | | 2.0 |
| 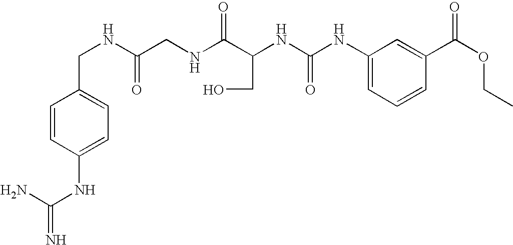 | WX-C312 | | | | 1.4 |
| 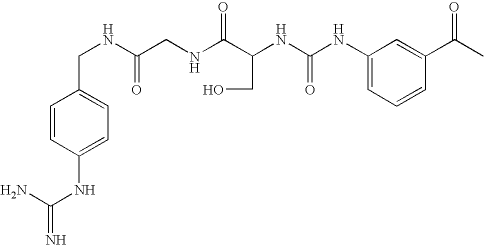 | WX-C314 | | | | no inh. *) |
| 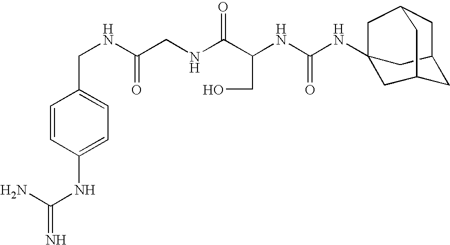 | WX-C320 | | | | >20 |
| 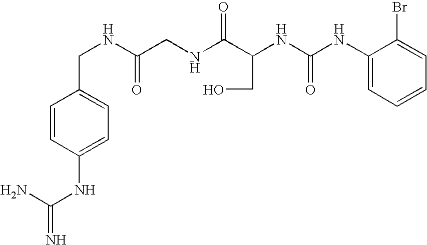 | WX-C322 | | | | >20 |
| 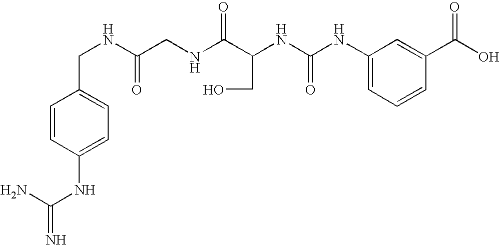 | WX-C324 | | | | 3.1 |
| 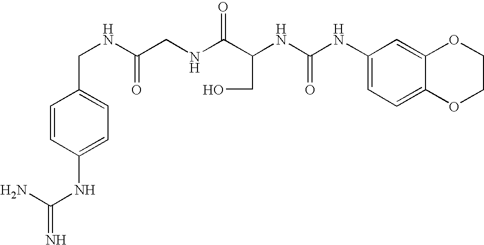 | WX-C326 | | | | >20 |
| 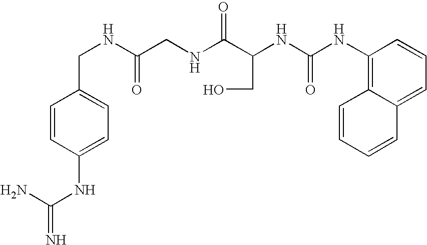 | WX-C328 | | | | >20 |
| 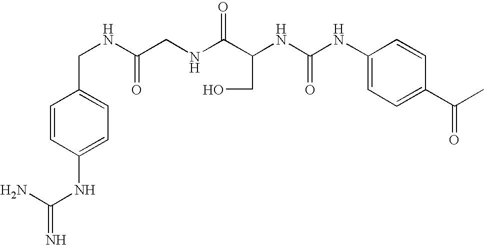 | WX-C330 | | | | >20 |
| 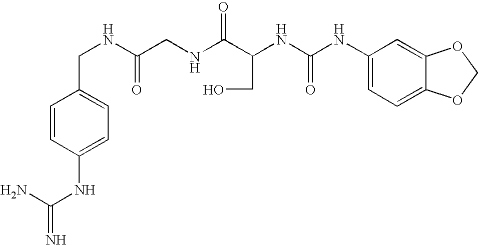 | WX-C332 | | | | >20 |
| 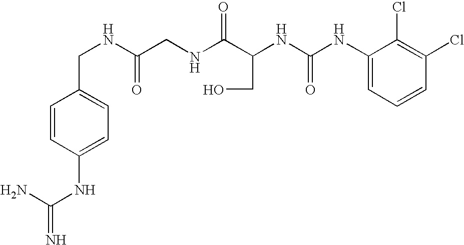 | WX-C334 | | | | >20 |
| 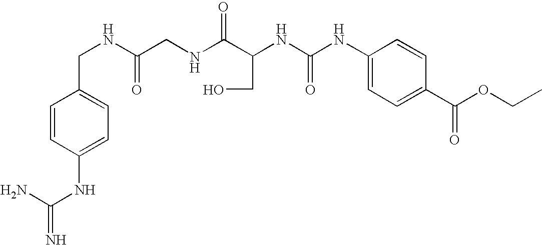 | WX-C336 | | | | 13.1 |
| 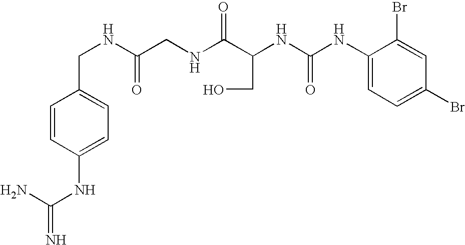 | WX-C338 | | | | >20 |
| 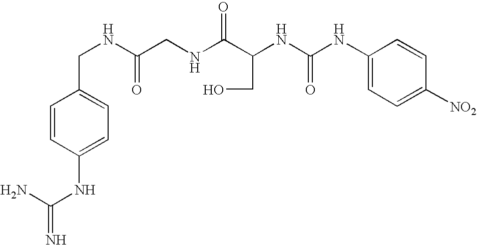 | WX-C342 | | | | 4.9 |
| 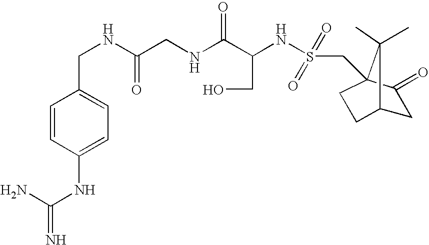 | WX-C296 | | | | 0.45 |
| 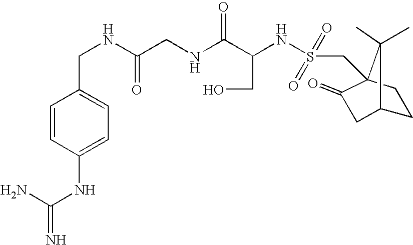 | WX-C298 | | | | 0.16 |
| 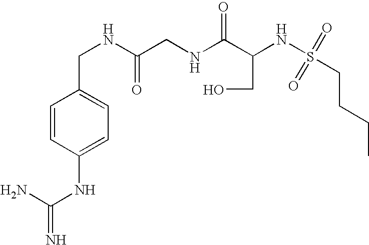 | WX-C300 | | | | 0.27 |
| 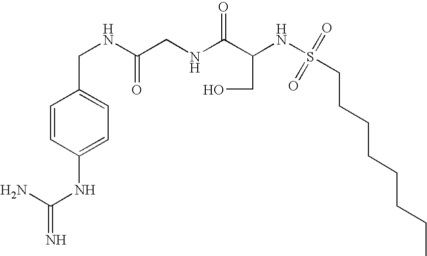 | WX-C302 | | | | 0.5 |
| 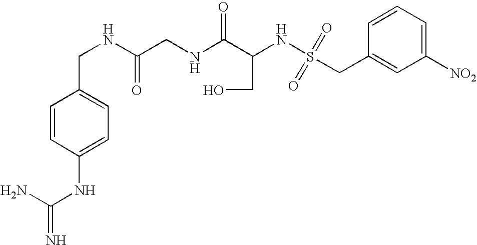 | WX-C316 | no inh. at 100 μM *) | no inh. at 100 μM *) | 32.4 | 0.09 |
| 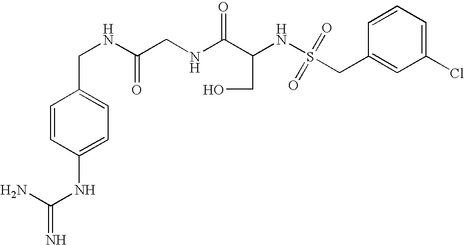 | WX-C318 | no inh. at 100 μM *) | no inh. at 100 μM *) | 36 | 0.047 |
| 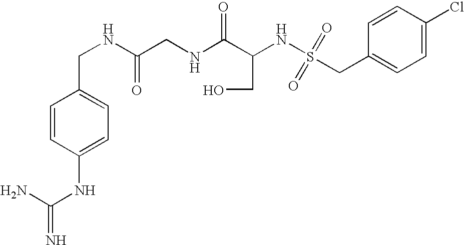 | WX-C340 | no inh. at 100 μM *) | no inh. at 100 μM *) | 34 | 0.01 |
| 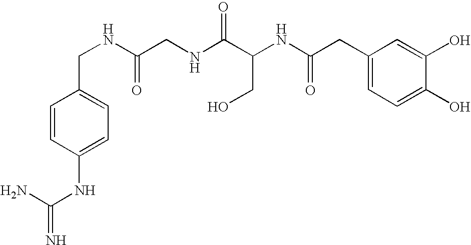 | WX-571 | | | | 14.7 |
| 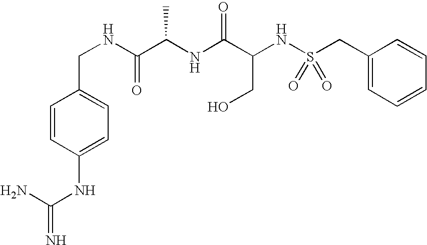 | WX-532 | 40 | no inh. at 100 μM *) | | 0.016 |
| 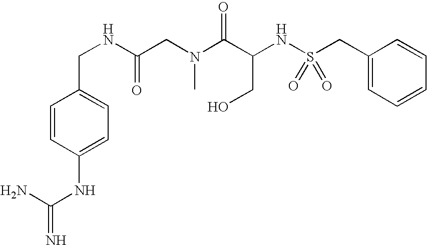 | WX-538 | 130 | >100 | >100 | 0.041 |
| 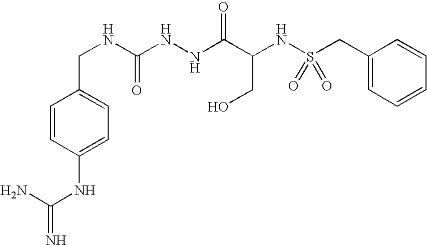 | WX-544 | | no inh. at 100 μM *) | | 0.83 |
| 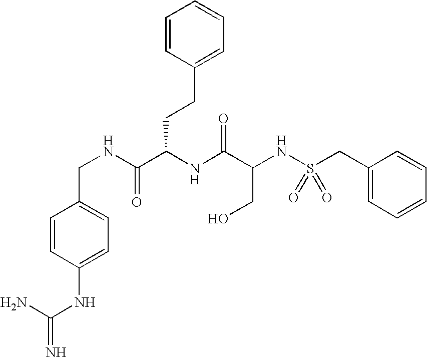 | WX-550 | 2.5 | >50 | >100 | 0.10 |
| 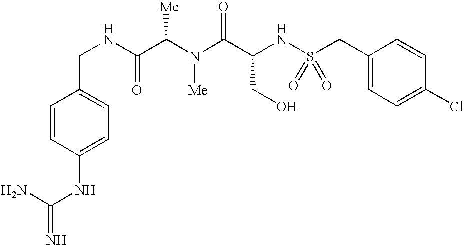 | WX-582 | | | | 0.01 |
| 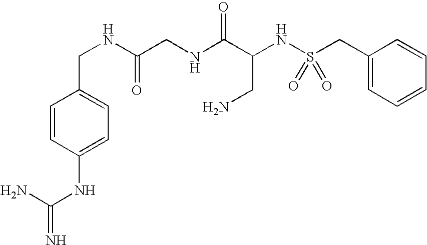 | WX-M6 | | | | 0.15 |
| 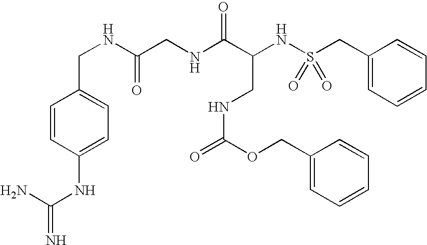 | WX-M5 | | | | 0.5 |
| 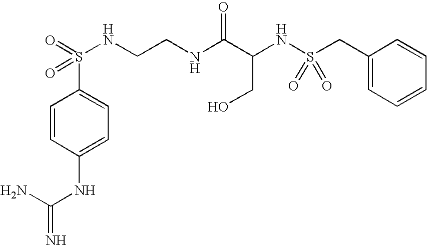 | WX-568 | | >1000 | >1000 | 2.1 |
| 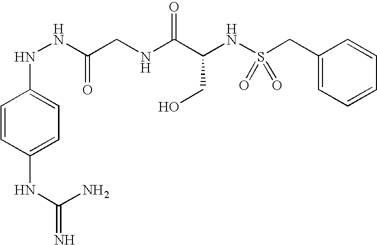 | WX-600 | | | | 2.1 |

*) no inh. = no inhibition
*) no inh. at = no inhibition at

### Example 15

In Vitro Inhibition of Urokinase by WX-508

In order to determine the uPA inhibitory activity, 200 μl Tris-buffer (0.05 mol/l containing the inhibitor, 0.154 mol/l NaCl, pH 8.0), 25 μl substrate (Pefachrome uPA or BZ-O-Ala-Gly-Arg-pNA in H₂O; Pentapharm Ltd., Basle, Switzerland) and 50 μl urokinase (Calbiochem-Novabiochem GmbH, Bad Soden, Germany) or another appropriate protease are incubated at 30° C. After ca. 30 minutes and 30 cycles the absorption is determined at 405 nm by means of a microplate reader (Mediators PHL, Aureon Biosystems, Vienna, Austria). The Kᵢ values were determined according to Dixon by linear regression using a computer program. The Kᵢ values are the mean of at least three determinations, the standard deviation was below 25%.

| | Kᵢ (μM) |
|---|---|
| urokinase | 0.04 |
| plasmin | >1000 |
| thrombin | >1000 |

### Example 16

Cell Culture Experiments (Caco-Assay)

Transport of the compounds WX-508, WXC-316, WXC-318, WXC-324, WXC-340, WX-532, WX-538, WX-550 and WX-582 across Caco-2 cell monolayers was investigated. In this experiment the bioavailability of the said compounds after oral administration was assessed.

16.1 Experimental Protocol

16.1.1 Cell Culture:

Caco-2-cells (American Type Culture Collection, Rockville, Md., USA) were cultured in Dulbecco's modified Eagle medium (DMEM) (Life Technologies, Gibco BRL, UK) containing 10% vol/vol heat-denatured foetal calf serum (FCS), 1% vol/vol non-essential amino acids, 160 U/ml benzylpenicillin and 100 U/ml streptomycin (Sigma Chemical, St. Louis, Mo., USA). The cells are kept at 37° C. in an atmosphere comprising 95% air and 5% CO₂ at 90% relative air humidity. The cells are cultured in 25 cm³ culture flasks, the medium being replaced every second day and the cells are trypsinized once per week.

16.1.2. Transport Experiments and Transepithelial Electric Resistance (TEER)

For the transport investigations the Caco-2 cells are cultured on porous polycarbonate filter membranes having a pore size of 0.4 μm and a surface of 4.7 cm² in clusters of 6 wells (Costar Transwell, Badhoevedorp, Netherlands). The cells are inoculated on each filter at an initial density of 10⁴ cells/cm². These cells are kept at 37° C. in one of the atmospheres described above. The medium is replaced every second day for a period of three weeks.

On the day of the transport experiment the culture medium was replaced by the same volume of Hank's balanced saline solution (HBSS) buffered with 30 mM HEPES at pH 7.2 (transport medium) and the cells are allowed to equilibrate for 1 hour. Afterwards the apical medium is replaced by 1.5 ml of a solution containing one of the compounds in HBSS/HEPES (10 μg/ml). Cell monolayers are incubated for four hours. Samples of the apical and basolateral compartments are removed at the end of the experiments and used to quantitatively determine the transport of the respective compounds.

The transepithelial electric resistance (TEER) is measured before and after equilibration using a Milicell ERS meter which was connected to a pair of electrodes in order to ensure the integrity of the monolayers formed on the filters. TEER is also measured at the end of the experiments to ensure that the cell monolayer remains coherent.

Permeability investigations on Caco-2-cell monolayers using ¹⁴C-mannitol are carried out by adding 1.5 ml of the radioactively labelled 1⁴C-mannitol solution in HBSS-HEPES (4 mmol/l with a specific activity of 0.2 μCi/ml) to the apical side.

Samples are taken from the apical and the basolateral side and analysed in a beta counter after adding a scintillation cocktail. All experiments were carried out three times.

The flow scheme of the experiment is as follows:

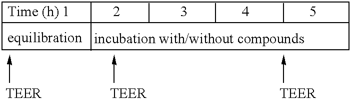

Results:

| Compound | % amount in the basolateral compartment | | |
|---|---|---|---|
| WX-508 | 0.2 | 0.1 | 0.7 |
| WXC-316 | 0.2 | 0.2 | 3.6 |
| WXC-318 | 0.4 | 1.0 | 4.5 |
| WXC-324 | 0.1 | 0.1 | 2.0 |
| WXC-340 | 2.4 | 0.2 | 0.1 |
| WX-532 | 0 | 0 | 0 |
| WX-538 | 0.2 | 0.2 | 0.1 |
| WX-550 | 0 | 3.1 | 0.6 |
| WX-582 | 0.4 | 3.1 | 5.2 |

16.1.3 Results

Caco-2-cell monolayers simulate the human intestinal absorptive epithelium and are a valuable tool for examining transepithelial transport. The TEER values are a control for the viability and coherence of the monolayer and were between 100% and 120%. TEER is defined as the product of resistance x surface. The resistance reflects the resistivity across dense connections (paracellular route) and not across cell membranes (transcellular route).

The values obtained in this in vitro transport investigation show that the examined compounds are transported from the apical side of the epithelial cell layer to the basolateral side. The results show that the investigated compounds have a bioavailability.

The results for the examined compounds are summarized again in the following table:

TABLE
Determination of the inhibitory constant, specificity and oral bioavailability

| | | Ki [μM] | Ki [μM] | Ki [μM] | Ki [μM] | Ki [μM] | Caco-2 assay |
|---|---|---|---|---|---|---|---|
| Formula | name | uPA | Plasmin | FXa | Thrombin | PK | % basolateral |
| 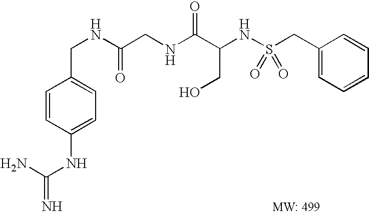 | WX-508 | 0.034 | >200 | | >350 | 180 | 0.2/0.1/0.7 |
| 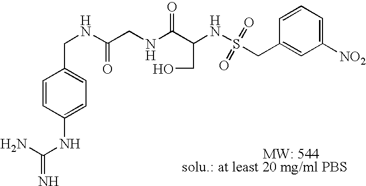 | WX-C316 | 0.09 | no inh. at 100 μM *) | 32.4 | no inh. at 100 μM *) | 875/ 0.2/0.2/3.4 0.1/0.1/3.6 | |
| 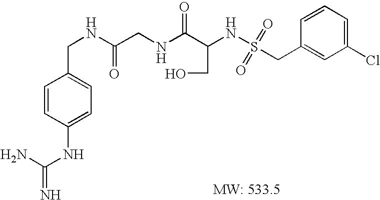 | WX-C318 | 0.047 | no inh. at 100 μM *) | 36 | no inh. at 100 μM *) | 176 | 0.4/1.0/4.5 0.2/0.6/7.0 |
| 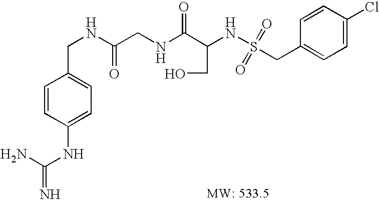 | WX-C340 | 0.01 | no inh. at 100 μM *) | 34 | no inh. at 100 μM *) | 216 | 2.4/0.2/0.1 2.3/0.1/0.1 |
| 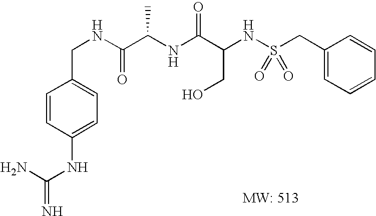 | WX-532 | 0.016 | 40 | | no inh. at 100 μM *) | 49 | 0/0/0 |
| 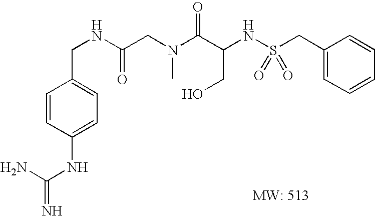 | WX-538 N-Me | 0.041 | 130 | >100 | >100 | 109 | 0.2/0.2/0.1 0.1/0.1/0 |
| 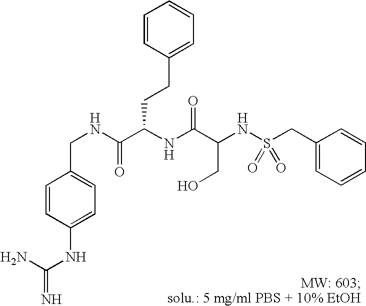 | WX-550 Homo- Phe | 0.10 | 2.5 | >100 | >50 | 268 | 0/3.1/0.6 |
| 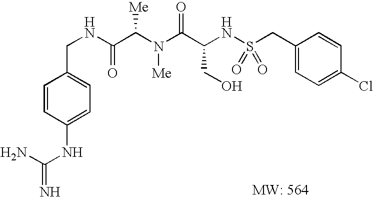 | WX-582 | 0.1 | | | | 58 | 0/3.1/5.2 0.4/2.9/3.0 |
| 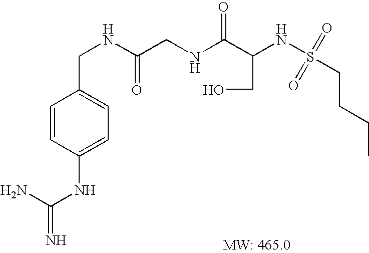 | WX-C300 | 0.27 | | | | | |
| 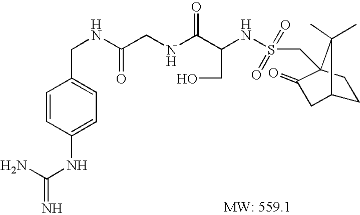 | WX-C298 | 0.16 | | | | | |
| | WX-C324 | | | | | | 0.1/0.1/2.9 0/0/3.0 |

*) no inh. = no inhibition
*) no inh. at = no inhibition at

### Example 17

### In Vivo Investigations

Ten inventive uPA inhibitor candidates were examined in rodents with respect to their oral bioavailability and plasma kinetics. These experiments were carried out on nine week old female Balb/c mice weighing between 17.6 and 18.5 g. The following test substances which are guanidinophenylalanine derivatives with HCl as the counterion were used:

| | | | Ki human |
|---|---|---|---|
| | | MW incl. HCl | uPA [μM] |
| 1 | WX-508 | 499 | 0.034 |
| 2 | WXC-316 | 544 | 0.09 |
| 3 | WXC-318 | 534 | 0.047 |
| 4 | WXC-340 | 533 | 0.01 |
| 5 | WX-532 | 513 | 0.016 |
| 6 | WX-538 | 513 | 0.041 |
| 7 | WX-550- | 603 | 0.10 |
| | Homo-Phe | | |
| 8 | WX-582 | 564 | 0.01 |
| 9 | WXC-300 | 465 | 0.27 |
| 10 | WXC-298 | 559 | 0.016 |

The compounds were used in a range from 0.1 to 1000 mg/kg, in particular 0.5 to 500 mg and most preferably 3 to 300 mg/kg. They were administered by means of a stomach tube. Samples were taken at 20, 40 and 60 minutes.

The results are summarized in the following table.

| | | | c [μg/ml] in | c [μg/ml] in |
|---|---|---|---|---|
| | Dose | time of sample | the serum | the serum |
| Test substance | mg/kg | collection | 1^{st} analysis | 2^{nd} analysis |
| WX-508 | 10 i.v. | 0 | 41.1 | 55.5 |
| | | 10 | 4.3 | 4.7 |
| | | 30 | 0.7 | 0.2 |
| | 30 oral | 20 | 0.3 | 0.3 |
| | | 40 | 5.7 | 4.5 |
| | | 60 | 0.08 | 0.09 |
| | 300 oral | 20 | 3.5 | — |
| | | 40 | 7.5 | 8.3 |
| | | 60 | 5.4 | 5.8 |
| WXC-316 | 10 i.v. | 0 | 45.4 | 67.5 |
| | | 10 | 12.4 | 14.3 |
| | | 30 | 2.7 | 2.6 |
| | 30 oral | 20 | 0.5 | 0.6 |
| | | 40 | 5.9 | 7.1 |
| | | 60 | 0.4 | 0.5 |
| | 300 oral | 20 | 6.0 | 7.2 |
| | | 40 | 21.8 | 32.1 |
| | | 60 | 5.3 | 5.8 |
| WXC-318 | 10 i.v. | 0 | — | 48.4 |
| | | 10 | 4.3 | 5.5 |
| | | 30 | 0.7 | 1.4 |
| | 30 oral | 20 | 0.1 | 0.2 |
| | | 40 | — | 0.05 |
| | | 60 | 0.2 | 0.2 |
| | 300 oral | 20 | 100 | 82.6 |
| | | 40 | 5.4 | 6.2 |
| | | 60 | 7.5 | 7.7 |
| WXC-340 | 10 i.v. | 0 | 51.8 | |
| | | 10 | 5.3 | |
| | | 30 | 1.7 | |
| | 30 oral | 20 | 0.3 | 0.5 |
| | | 40 | 0.3 | 0.8 |
| | | 60 | 1.4 | 2.3 |
| | 300 oral | 20 | 4.5 | |
| | | 40 | 4.9 | |
| | | 60 | 4.3 | |
| WX-532 | 10 i.v. | 0 | 61.7 | 68.8 |
| | | 10 | 3.8 | 3.5 |
| | | 30 | 11.4 | 11.2 |
| | 30 oral | 20 | 1.5 | |
| | | 40 | 10.9 | |
| | | 60 | 0.9 | |
| | 300 oral | 20 | 5.8 | 6.7 |
| | | 40 | 2.9 | 3.3 |
| | | 60 | 12.8 | 15.0 |
| WX-538 | 10 i.v. | 0 | 45.8 | |
| | | 10 | 5.5 | |
| | | 30 | 0.6 | |
| | 30 oral | 20 | 0.1 | |
| | | 40 | 0.05 | |
| | | 60 | 0.2 | |
| | 300 oral | 20 | 2.5 | |
| | | 40 | 3.5 | |
| | | 60 | 2.3 | |
| WX-550 | 2.5 i.v. | 0 | 7.2 | |
| | | 10 | 0.7 | |
| | | 30 | 0.02 | |
| | 7.5 oral | 20 | 0 | |
| | | 40 | 0 | |
| | | 60 | 0 | |
| | 75 oral | 20 | 0 | |
| | | 40 | 0.3 | |
| | | 60 | 0 | |
| WX-582 | 2.5 i.v. | 0 | 11.5 | |
| | | 10 | 0.8 | |
| | | 30 | 0.1 | |
| | 7.5 oral | 20 | 0 | |
| | | 40 | 0 | |
| | | 60 | 0 | |
| | 75 oral | 20 | 1 | |
| | | 40 | 0.7 | |
| | | 60 | 0.4 | |
| WXC-298 | 10 i.v. | 0 | 66.2 | |
| | | 10 | 18.3 | |
| | | 30 | 2.1 | |
| | 30 oral | 20 | 0.2 | |
| | | 40 | 0.4 | |
| | | 60 | 0.1 | |
| | 300 oral | 20 | 17.0 | |
| | | 40 | 10.7 | |
| | | 60 | 7.0 | |
| WXC-300 | 10 i.v. | 0 | 94.4 | |
| | | 10 | 11.0 | |
| | | 30 | 1.3 | |
| | 30 oral | 20 | 0.2 | |
| | | 40 | 0.2 | |
| | | 60 | 0.2 | |
| | 300 oral | 20 | 1.5 | |
| | | 40 | 4.6 | |
| | | 60 | 0.6 | |

### Example 18

In Vivo Assay of the uPA Inhibitor for Tumor Spreading, Tumor Growth and Metastasizing in a Rat Breast Cancer Model

10-25 mm³ of transplantable metastasising mammary carcinoma BN-472 breast cancer tumor fragments from donor rats were transplanted orthotopically under the mammary fat pad. (Day 0) into 6 to 8 weeks old female brown Norwegian rats. Subcutaneous treatment was started 5 days after tumor inoculation. Each group consisted of 18 animals. The treatment group received a daily dose of 0.3 mg/kg body weight subcutaneously. The treatment-was carried out over a period of 4 weeks.

The dimensions of the subcutaneous tumors and the weight of the animals were determined weekly. At the end of the treatment the animals were sacrificed and tumor weights, organ weights and the number of metastases in relevant tissues were determined.

Statistics

The data were analyzed using the SigmaStat Vers.2.03 statistical package. Tumor sizes, tumor weights and the weights of the organs were checked with the Shapiro-Wilk W-test for normality of distribution (Royston P., Approximating the Shapiro-Wilk W-test for non-normality. Statistics and Computing 1992, 2:117-119). For each parameter the Shapiro-Wilk W-tests were performed on all data (e.g., tumor weights of all rats), and separately for each treatment group. If one of the P-values was <0.05 the variable was considered to be abnormally distributed and the Mann-Whitney U-test was used to test for significance levels of differences. The Mann-Whitney U-test was used to test for differences in weights of the axillary lymph nodes, intra peritoneal lymph nodes, tumor, and the number of lung foci. Primary and metastatic tumor data at the end of the treatment period were graphically represented using the Box-Whisker graphs, with the box showing the inter-quartile range of values, with a horizontal line at the median value. Whiskers represent the 5^{th} and 95^{th} percentile of values, and the individual values are indicated by dots. The unadjusted level for statistical significance was α=0.05. All tests were two-sided.

Summary of the Results

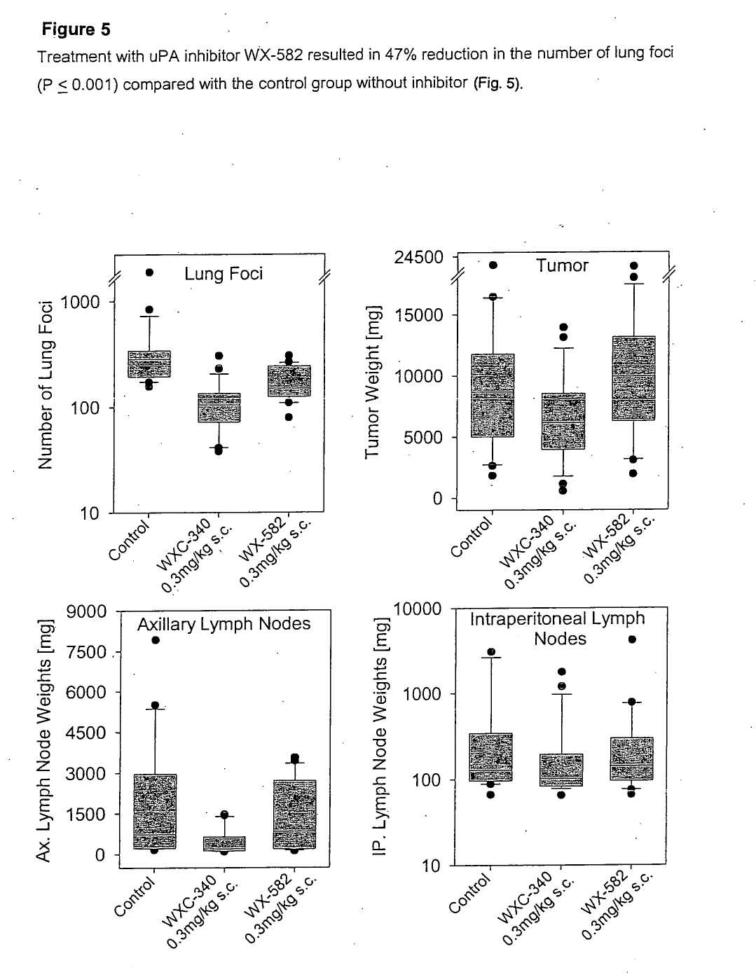

Treatment with uPA inhibitor WXC-340 resulted in 63% reduction in the number of lung foci (P≦0.001) and 73% reduction of the axillary lymph node weights (P=0.006) compared with the control group without inhibitor (FIG. 5). The median tumor weight was reduced by 29% (P=0.141)

1. Use of compounds of the general formula I 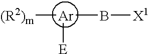
in which
Ar denotes an aromatic or heteroaromatic ring system,
E denotes 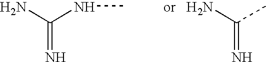
or Ar and E together form a residue 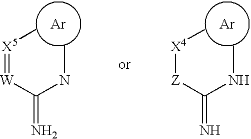
in which Z can be O, NH or C═O and X⁴ can be C═O, NH or CH₂ and W can be N, CR³ or CR⁶ and X⁵ can be CH, CR³, CR⁶ or N,
B denotes —SO²—, —CR³₂—, —NR³— or —NH—,
X¹ denotes NR³R⁴ OR³ SR³ COOR³ CONR³R⁴ or COR⁵,
R¹ denotes H, an optionally substituted alkyl, alkenyl, alkinyl, aryl, heteroaryl residue or COOR³, CONR³R⁴ or COR⁵,
R² denotes halogen, C(R⁶)₃, C₂(R⁶)₅, OC(R⁶)₃ or OC₂(R⁶)₅,
R³ denotes H or any organic residue,
R¹³ denotes a group of the general formula (IIa) or (IIb), 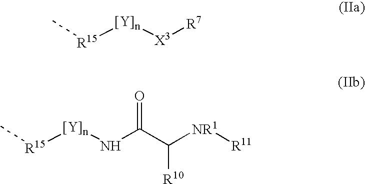
X² denotes NH, NR⁴, O or S,
X³ denotes NH, NR4, O, S, CO, COO, CONH or CONR⁴,
Y denotes C(R⁸)₂, NH or NR³,
R⁴ denotes H or a branched or unbranched, optionally substituted alkyl, alkenyl or alkinyl residue,
R⁵ denotes H, an alkyl, alkenyl, alkinyl, carboxy-alkyl, carboxy-alkenyl, carboxyl alkinyl, carboxy-aryl, carboxy-heteroaryl, —(CO)NR³R⁴ or —COO—R³ in which the alkyl, aryl and heteroaryl residues can optionally be substituted,
R⁶ is in each case independently H or halogen and in particular F,
R⁷ denotes H or an optionally substituted alkyl, alkenyl, alkinyl, aryl or heteroaryl residue or —COR⁹,
R⁸ in each case independently denotes H, or a branched or unbranched, optionally substituted alkyl, alkenyl, alkinyl, aryl, heteroaryl, aralkyl, alkylaryl, heteroaralkyl residue or/and a substituted or unsubstituted bicyclic or polycyclic residue,
R⁹ denotes H or a branched or unbranched, optionally substituted alkyl, alkenyl, alkinyl, aryl or/and heteroaryl residue,
R¹⁰ denotes a residue (C(R¹)₂)ₒ—X³R⁵,
R¹¹ denotes H, a carbonyl residue —CO—R², a carbonamido residue —CONR²₂,
an oxycarbonyl residue —COO—R¹² or particularly preferably a sulfonyl residue —SO₂R¹²,
R¹² denotes H, a branched or unbranched, substituted or unsubstituted alkyl, alkenyl, alkinyl, aryl or heteroaryl residue or a substituted or unsubstituted cyclic alkyl residue or a substituted or unsubstituted aralkyl, alkylaryl or heteroaralkyl residue or a substituted or unsubstituted bicylic or polycyclic residue,
R¹⁵ represents C═X², NR³ or CR³₂,
n is an integer from 0 to 2,
m is an integer from 0 to 5,
o is an integer from 1 to 5,
p is an integer from 1 to 5,
or salts of these compounds to produce an agent for inhibiting the urokinase plasminogen activator.

2. Use as claimed in claim 1 of compounds of the general formula III 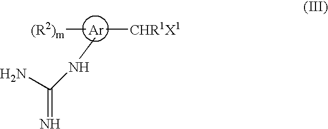
in which
Ar denotes an aromatic or heteroaromatic ring system,
X¹ denotes NR³R⁴, OR³, SR³, COOR³, CONR³R⁴ or COR⁵,
R¹ denotes H, an optionally substituted alkyl, alkenyl, alkinyl, aryl, heteroaryl residue or COOR³, CONR³R⁴ or COR⁵,
R² denotes halogen, C(R⁶)₃, C₂(R⁶)₅, OC(R⁶)₃ or OC₂(R⁶)₅,
R³ denotes H or any organic residue,
R¹³ denotes a group of the general formula (IVa) or (IVb), 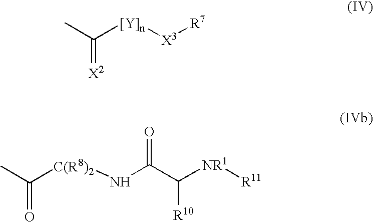
X² denotes NH, NR⁴, O or S,
X³ denotes NH, NR⁴, O, S, CO, COO, CONH or CONR⁴,
Y denotes C(R⁸)₂, NH or NR³,
R⁴ denotes H or a branched or unbranched, optionally substituted alkyl, alkenyl or alkinyl residue,
R⁵ denotes H, an alky, alkeny, alkiny, carboxy-alky, carboxy-alkenyl, carboxyl alkinyl, carboxy-aryl, carboxy-heteroaryl, —(CO)NR³R⁴ or —COO—R³ in which the alkyl, aryl and heteroaryl residues can optionally be substituted,
R⁶ is in each case independently H or halogen and in particular F,
R⁷ denotes H or an optionally substituted alkyl, alkeny, alkiny, aryl or heteroaryl residue or —COR⁹,
R⁸ in each case independently denotes H, halogen, or a branched or unbranched, optionally substituted alkyl, alkinyl, aryl, heteroaryl residue or/and (CH₂)ₘ—OH,
R⁹ denotes H or a branched or unbranched, optionally substituted alkyl, alkenyl, alkinyl, aryl or/and heteroaryl residue,
R¹⁰ denotes a residue (C(R¹)₂)ₒ—X³R⁵,
R¹¹ denotes H, a carbonyl residue —CO—R¹², an oxycarbonyl residue —COO—R¹² or particularly preferably a sulfonyl residue —SO₂R¹², R¹² denotes a branched or unbranched, substituted or unsubstituted alkyl, alkenyl, alkinyl, aryl or heteroaryl residue or a substituted or unsubstituted cyclic alkyl residue or a substituted or unsubstituted aralkyl, alkylaryl or heteroaralkyl residue or a substituted or unsubstituted bicylic or polycyclic residue,
n is an integer from 0 to 2,
m is an integer from 0 to 5,
o is an integer from 1 to 5,
or salts of these compounds to produce an agent for inhibiting the urokinase plasminogen activator.

3. Use as claimed in claim 1 wherein Ar denotes a benzene ring.

4. Use as claimed in claim 1, wherein R¹² denotes a benzyl residue.

5. Use as claimed in claim 1, wherein R¹² denotes an adamantyl or camphor residue.

6. Use as claimed in claim 1, wherein the substituents B in particular-CHX¹R¹ and E, in particular —NHC(NH)NH₂, are arranged in a para position relative to one another.

7. Use as claimed in claim 1, wherein the compound N-[2-(4 guanidino-benzenesulfonyl-amino)-ethyl]-3-hydroxy-2-phenylmethanesulfonylamino-propionamide hydrochloride, Bz-SO₂-(D)-Ser-(Aza-Gly)-4-guanidino-benzylamide hydrochloride, N-(4-guanidino-benzyl)-2-(3 hydroxy-2-phenylmethane-sulfonylamino-propionylamino)-4-phenyl butyramide hydrochloride, N-[(4-guanidino-benzylcarbamoyl)-methyl]-3-hydroxy-2-phenylmethanesulfonylaminopropionamide, 3-nitrobenzyl-sulfonyl-(D)-Ser-Gly-(4-guanidinobenzyl)amide hydrochloride (WXC-316), 3-chlorobenzyl-sulfonyl-(D)-Ser-Gly-(4-guanidinobenzyl)amide hydrochloride (WXC-318), 4-chlorobenzyl-sulfonyl-(D)-Ser-Gly-(4-guanidinobenzyl)amide hydrochloride (WXC-340), benzylsulfonyl-(D)-Ser-Ala-(4-guanidinobenzyl)amide hydrochloride (WX-532), 4-chlorobenzylsulfonyl-(D)-Ser-N-Me-Ala-(4-guanidinobenzyl)amide (WX-582) or benzylsulfonyl-(D)-Ser-N-Me-Gly-(4-guanidinobenzyl)amide (WX-538) is used.

8. Use as claimed in claim 1 to combat diseases that are associated with a pathological overexpression of urokinase or/and urokinase receptor.

9. Use as claimed in claim 1 to combat tumors.

10. Use as claimed in claim 1 to combat the formation of metastases.

11. Use as claimed in claim 1, wherein a pharmaceutical preparation is produced that can be administered orally, topically, rectally, parenterally, subcutaneously, intramuscularly, intraperitoneally, sublingually, nasally or by inhalation.

12. Use as claimed in claim 1, wherein the agent is produced in the form of tablets, dragees, capsules, pellets, suppositories, solutions, emulsions, suspensions, liposomes, inhalation sprays or transdermal systems such as plasters.

13. Compounds of formula I 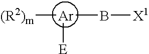
wherein Ar, X¹, R², B, E and m are defined as in claim 1.

14. Compounds of formula III 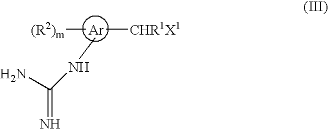
wherein Ar, X¹, R¹, R² and m are defined as in claim 2.

15. N-[2-(4-guanidino-benzenesulfonyl-amino)-ethyl]-3-hydroxy-2-phenylmethanesulfonylamino-propionamide hydrochloride, Bz-SO₂-(D)-Ser-(Aza-Gly)-4-guanidino-benzylamide hydrochloride, N-(4-guanidino-benzyl)-2-(3-hydroxy-2-phenylmethane-sulfonylamino-proprionylamino)-4-phenylbutyramide hydrochloride, N-[(4-guanidino-benzylcarbamoyl)-methyl]-3-hydroxy-2-phenylmethanesulfonylaminopropionamide or 3-nitrobenzylsulfonyl-(D)-Ser-Gly-(4-guanidinobenzyl)amide hydrochloride (WXC-316), 3-chlorobenzyl-sulfonyl-(D)-Ser-Gly-(4-guanidinobenzyl)amide hydrochloride (WXC-318), 4-chlorobenzyl-sulfonyl-(D)-Ser-Gly-(4-guanidinobenzyl)amide hydrochloride (WXC-340), benzylsulfonyl-(D)-Ser-Ala-(4-guanidinobenzyl)amide hydrochloride (WX-532), 4-chlorobenzylsulfonyl(D)-Ser-N-Me-Ala-(4-guanidinobenzyl)amide (WX-582) or benzylsulfonyl(D)-Ser-N-Me-Gly-(4-guanidinobenzylamide (WX-538).

16. Method for inhibiting urokinase in living organisms by administering an effective amount of at least one compound as claimed in claim 13.

17. Method for inhibiting urokinase in humans by administering an effective amount of at least one compound as claimed in claim 13.

18. Pharmaceutical preparation containing a therapeutically active amount of a compound as claimed in claim 13.
